(19) United States
(12) Patent Application Publication
(10) Pub. No.: US 2010/0111983 A1
(43) Pub. Date: May 6, 2010

(54) Method for Using Lowstrength Electric Field Network (LSEN) and Immunosuppressive Strategies to Mediate Immune Responses

(75) Inventor: Luyi Sen, Stevenson Ranch, CA (US)

Correspondence Address: Law Offices of Daniel L. Dawes;Dawes Patent Law Group, 5200 Warner Blvd, Ste. 106, Huntington Beach, CA, 92649, US

(73) Assignee: THE REGENTS OF THE UNIVERSITY OF CALIFORNIA, Oakland, CA (US)

(21) Appl. No.: 12/531,291

(22) PCT Filed: Mar. 12, 2008
(86) PCT No.: PCT/US08/56603

§ 371 Date: Sep. 17, 2009

Related U.S. Application Data
(60) Provisional application No. 60/894,831, filed on Mar. 14, 2007.

(51) Int. Cl. A61K 39/00; A01N 1/00; C12N 13/00; C12M 1/00

(52) U.S. Cl. 424/184.1; 435/1.1; 435/173.1; 435/283.1

(57) ABSTRACT

Application to an allograft or xenograft of a low strength electric field network (LSEN) together with an immuno-suppressive drug, gene and siRNA or other gene-based therapy is used to mediate the immune responses within an donor organ, tissue or cells, to prevent the acute and chronic rejection and to induce true tolerance, The gene(s) is locally transferred ex vivo in the time interval between harvest and implantation of allografts or xenografts before the implantation to introduce the long-term over expression of immunosuppressive and/or modulative molecules, or for down regulating alloreactive molecules in the donor organ, tissue or cells only and not in the recipient's whole body system.

### RELATED APPLICATIONS

The present application is related to U.S. Provisional Patent Application Ser. No. 60/894,831, filed on Mar. 14, 2007, which is incorporated herein by reference and to which priority is claimed pursuant to 35 USC 119.

### BACKGROUND OF THE INVENTION

1. Field of the Invention

The invention relates to a methodology for using low strength electric field network (LSEN) electropermeabilization to mediate immune responses within a donor organ, tissue or cells to prevent rejection and to induce true tolerance.

2. Description of the Prior Art

Allograft rejection remains a major obstacle to successful heart transplantation. Immunosuppression can be induced by administering one or several types of pharmacologic agents. However, systemic immunosuppression usually results in multiple, deleterious side effects requiring major dosage adjustments, and true tolerance is rarely achieved. The annual cost for heart transplants in United States is over $25 billion per year in 2002. This cost is increases substantially every year due to the requirement of life-long immunosuppressive therapy, rehospitalization and retransplantation. Immunosuppressive drugs accounts for approximately 60% of the routine costs. Acute rejection occurs in 75% of human cardiac allografts within the first 6 months after transplantation, and is characterized by a monocyte and cytotoxic T lymphocytes infiltration. Chronic rejection occurs in 50% of heart allografts and it is a major limitation to long-term graft success and the patients' survival.

The most devastating manifestation of chronic rejection in cardiac allografts presents as a diffuse intimal proliferative arteriosclerotic process, a disease was known as allograft coronary vasculopathy. The initiating event in the development of chronic rejection is not known. Although hyperacute and acute rejection have largely been ameliorated with the use of pre-transplantation cross-matching and immunosuppression, allograft rejection and the side effects of immunosuppressive regimens are still the main cause of rehospitalization, retransplantation, morbidity and early mortality.

Improvements in immunosuppression will need to rely on more specific interventions at the level of lymphocyte priming and activation. Additional measures will require that immunomodulatory signals be delivered specifically to regions of antigen presentation to avoid systemic immunosuppression or other metabolic deficits.

The time interval between harvest and implantation of cardiac transplants provides a unique temporal opportunity to biologically modify the graft ex vivo and to pave the way for local or organ-specific immunosuppression. Soluble proteins secreted within the transplanted organ may act locally and specifically while avoiding systemic side effects and avoiding the need for conventional systemic immunosuppression. This strategy, however, depends on efficient and stable gene transfer into the whole target organ.

Viral vectors are so far the most efficient tool for delivery of genes in to mammalian cells and currently dominate gene therapy clinical trials. Adenoviral vectors can introduce foreign genes into differentiated nondividing cells in living animal tissues. Given the specific attributes of nondividing cardiac cells, mutant adenoviral vectors emerged as the most effective vehicle for transport of genes into the heart under both normothermic and hypothermic conditions. However, the foreign genes are only expressed transiently. Recombinant adeno-associated virus has a tropism for many mammalian cell types and has the capacity for integration into the host genome, thereby permitting long-term expression. Although, the gene transfer efficiency is high, nevertheless, so far all attenuated viruses have potential toxicity, and immunogenecity to prevent long-term expression and repeated use.

Cationic lipids are widely used as a nonviral vector for gene transfer in vitro. They are both inexpensive and readily available. By virtue of their positive charge, they spontaneously associate with the negatively charged plasmid DNA to form a stable cationic lipid-DNA complex that facilitates DNA transfer into the cell. Unlike certain viral vectors, they do not generate an immune response and they also eliminate the risk of recombination or complementation. While cationic liposomes have the ability to transport reporter and therapeutic genes into the cardiac myocytes, the efficiency of this vector is considerably higher than naked plasmid DNA, but still 5 to 15 times lower than adenovirus that often limits the therapeutic efficacy.

Mutant adenoviral vectors emerged as the most effective vehicle for transport of a gene into the heart under both normothermic and hypothermic conditions. However, the toxicity, immunogenecity and extensive ectopic transfection of virus-mediated gene transfer has led to a major setback for gene therapy clinical trials. In liposome-mediated gene transfer, although non-toxic and repeatedly usable, the gene transfer efficiency is so low that it often limits its therapeutic efficacy. Electroporation is a technique involving the application of short duration, high intensity (200-1500 Volt/cm) electric field pulses to cells. However, more than 10 kV is needed to electropermeabilize the large animal or human heart if we use conventional 2-6 needle electrodes or a pair of plate electrodes. Electroporation is a known effective technique and is commonly used for in vitro gene transfection of cell lines and primary cultures, but a limited amount of work has been reported in small animal organs and tissues. The efficiency of electroporation-mediated gene transfer is higher than any viruses. However, the requirement of the high voltage limits its application in large animal and human organs.

### BRIEF SUMMARY OF THE INVENTION

The illustrated embodiment of the invention is directed to a methodology for using a combination of a highly efficient low strength electric field network (LSEN) and an immunosuppressive drug, gene and siRNA or other gene-based therapy to mediate the immune responses within an donor organ, tissue or cells to prevent the acute and chronic rejection and to induce true tolerance. The time interval between harvest and implantation of allografts or xenografts provides a unique opportunity for locally transferring gene(s) ex vivo before the implantation to introduce the long-term over expression of immunosuppressive and/or modulative molecules, or for down regulating alloreactive molecules in the donor organ, tissue or cells only and not in the recipient's whole body system. Locally transferring or down regulating acts as immune system mask or suppressant on the donor organ, tissue or cells and greatly increases the therapeutic efficacy, limit systemic side effects.

In addition to application of LSEN, the illustrated embodiment of the invention includes two elements: 1) local delivery of the combination of drug, gene and siRNA or other gene-based therapeutic molecules to the donor organ, tissue or cells; and 2) combinations of drug, gene and siRNA or other gene-based therapy molecules used to modulate the immune responses within an donor organ, tissue or cells to prevent the acute and chronic rejection and induce true tolerance in transplantation.

In the specification where it is stated that at least one molecule or gene is used in some manner, it is to be understood that this is to be taken to mean that at least one kind of molecule or gene is so utilized in an amount sufficient to be efficacious for the desired result. It is of course very unlikely that utilization of a single molecule or gene will be sufficient to cause any practical bioeffect in an organ, tissues or a plurality of cells.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIGS. 1a-1d are a sequence of diagrams illustrating the illustrated embodiment wherein an application of LSEN is made to the heart ex vivo.

FIG. 1a is comprised of three illustrations from left to right of a cross sectional view of a heart in which unexpanded LSEN baskets have been deployed, of a plan view of a heart on whose surface an LSEN mesh has been deployed and of a cross sectional view of a heart in which expanded LSEN baskets have been deployed with an inset in enlarged scale of the myocardial wall diagrammatically illustrating the LSEN fields.

FIG. 1b is a diagram of the plasmid which is infused.

FIG. 1c is a timing diagram of the waveform of the voltage bursts applied between the LSEN baskets and mesh in FIG. 1a.

FIG. 1d is a diagram illustrating the cervical heterotopic functional heart implant model used in the illustrated embodiment as part of the proof of concept.

FIG. 2a is a comparative graph of the transgene/GAPDH expression ratio for various prior art for gene transfer methods, adenovirus-mediated IL-10 gene transfer (Adv-IL-10), cationic liposome-mediated IL-10 (Lip-II-10) gene transfer, used in the same heart transplant model, and the illustrated embodiment as well as a control as a function of postoperative days (POD).

FIG. 2b are two comparative histological microphotographs of myocardium of a control and the illustrated embodiment.

FIG. 2c is a bar chart of transfection efficiency in percentage for the illustrated embodiment as compared to two prior art methods, adenovirus-mediated (Ave-IL10) or liposome-mediated (Lip-IL10) gene transfer.

FIG. 2d is a bar chart of the transgene/GAPDH expression ratio for various locations in the heart, namely the left and right ventricles LV, RV; the left and right atria LA, RA; and the interventricular septum IVS.

FIG. 2e are photographs of a portion of gels showing localizations in the two donor hearts (DN), and recipients' brain (B), lung (L), heart (RH) and skeletal muscle (SM) resulting the method of the illustrated embodiment compared to two prior art methods, adenovirus and liposomes.

FIG. 2f are photographs of a gel showing the level of LSEN-mediated transgene over expression induced IL-10 protein expression, the product of IL-10 gene expression, in the left ventricular myocardium of donor hearts in comparison with that in adenovirus-phIL-10 group and liposome-phIL-10 group.

FIG. 2g is a comparative bar chart of the IL-10 protein expression of the illustrated embodiment compared to two prior art methods as a function of POD.

FIG. 3a is a comparative graph of the transgene/GAPDH expression ratio as a function of the LSEN field strength in V/cm.

FIG. 3b is a comparative graph of the transgene/GAPDH expression ratio resulted in several different LSEN field strength in V/cm as a function of POD.

FIG. 3c are photographs of a portion of gels showing specific increase in IL-10 protein expression induced by gene transfer in the left ventricular myocardium of donor hearts for several different LSEN field strengths, but the Actin, a heart native control protein expression was not changed.

FIG. 3d is a comparative histological microphotograph of myocardium taken after two different LSEN field strength applications.

FIG. 3e is a bar chart of the transgene/GAPDH expression ratio as a function of pulse duration of the LSEN field.

FIG. 3f is a bar chart of the transgene/GAPDH expression ratio as a function of pulse interval of the LSEN field.

FIG. 3g is a bar chart of the transgene/GAPDH expression ratio as a function of burst number of the LSEN field.

FIG. 3h is a bar chart of the transgene/GAPDH expression ratio as a function of interburst interval of the LSEN field.

FIG. 3i is a bar chart of the transgene/GAPDH expression ratio as a function of the number of pulses per burst in the LSEN field.

FIG. 4a is a comparative graph of the left ventricular endomyocardium monophasic action potential for a control and 10 v/cm LSEN treated implanted heart; and a bar chart showing the endomyocardium monophasic action potential duration at 90% of repolarization (APD₉₀) for the illustrated embodiments compared to a control group C and various prior art methods.

FIG. 4b is a comparative bar chart of the amplitude Vmax of the action potential stroke for a control group, two groups treated by prior art methods and the illustrated embodiment at three different LSEN field strengths.

FIG. 4c is a bar chart the number of cases in percentages of atrial and ventricular arrhythmias, namely supraventricular tachycardia SVT, ventricular tachycardia VT, atrial fibrillation AF, ventricular fibrillation VF.

FIG. 4d is a comparative bar chart of left ventricular peak systolic pressure in mmHg for a control group, and groups treated with the illustrated embodiments and two prior art methods.

FIG. 4e is a comparative bar chart of the dV/dt of the left ventricular peak systolic pressure in mmHg for a control group, and groups treated with the illustrated embodiments and two prior art methods.

FIG. 5a is a graph of the cardiac allograft survival as a percentage as a function of POD for a group treated according to the illustrated embodiment, a control group and two prior art methods.

FIG. 5b is a series of histological microphotographs of myocardium for a group treated according to the illustrated embodiment, a control group and two prior art methods at different postoperative days.

FIG. 5c is a comparative bar chart of left ventricular peak systolic pressure in mmHg for a control group, and groups treated with the illustrated embodiments (LSEN-IL-10), LSEN only, and two prior art methods compared with that in control (allografts) and recipients' native heart at POD 8, and treated with the illustrated embodiments (LSEN-IL-10) compared with liposome-IL-10 at POD 28.

FIG. 6a is a data graph showing the amount of IL-10 and β-actin gene expression in cardiac allografts treated with hIL-4 and hIL-10 combinatorial gene-transfer.

FIG. 6b is a comparative bar chart of the time-course of IL-10 transgene expression in the cardiac allografts in hIL-4 and hIL-10 combinatorial gene-transfer.

FIG. 6c is a comparative bar chart of the gene transfer efficiency in percentages for efficiency of LSEN-mediated ex vivo hIL-4 and IL-10 combined gene transfer in cardiac allograft evaluated by in situ hybridization of the anti-sense and sense digoxygenin-labeled riboprobes of hIL-4 and IL-10 mRNA.

FIG. 6d is a comparative bar chart of the IL4/IL10 protein expression at different heart locations, namely LV, IVS, RV, LA and RA.

FIG. 7a is graph of cardiac allograft survival in percentages as a function of POD for a control and LSEN-IL-10 and hIL-4 and IL-10 combined gene therapy (LSEN-IL-10 and IL-4) groups.

FIG. 7b is a comparative bar chart of the graft infiltrating cells for the total infiltrating cells and CD3+ T cells in a control group, a group treated with LSEN-IL4 and IL10 combined gene therapy and a group treated with LSEN-IL10.

FIG. 7c is a comparative bar chart of the left ventricle dP/dt for LSEN-IL-10 and LSEN-IL-4-IL10 groups compared with that in control group (treated with saline) and recipients' native hearts (isograft).

FIG. 8a is a comparative graph of the gene expression level as a function of POD for a CTLA4-Ig group and IL-10 group.

FIG. 8b is a comparative bar chart of the reduction of the total number of infiltrating lymphocytes induced by five different therapy groups.

FIG. 8c is a comparative bar chart of the number of indefinitely surviving allografts in percentages as a function of four different therapy groups.

FIG. 9 is a schematic diagram of the transfer CTLA4Ig gene and CD40Ig gene to block two major co-stimulatory pathways, and CIITA-siRNA to down regulate MCHII expression in the cardiac allograft.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The illustrated embodiment of the invention is a major breakthrough using a highly efficient and safe, low strength electric field network (LSEN)-mediated gene transfer approach for ex vivo gene transfer in the whole heart of large animal and human. The illustrated embodiment of the invention enables us to use a very low strength (≦10 v/cm) electric field network (LSEN) to induce a fast and highly efficient naked plasmid DNA transfer in whole heart of large animal and human. LSEN meshes and electropermeabilization methodologies are disclosed in Provisional Patent Application Ser. No. 60/744,522, filed: Apr. 10, 2006 and Provisional Patent Application Ser. No. 60/819,277, filed: Jul. 6, 2006, both of which are incorporated herein by reference (hereinafter called LSEN). The devices and signal protocols used in LSEN will not be further described in detail other where necessary to provide contextual background. LSEN is more properly referred to as a low strength electropermeabilizing field network rather than low strength electropermeabilizing field network, because at the low voltage levels which LSEN uses the biomechanism is believed to be qualitatively different than in conventional high voltage electroporation. It is currently understood that LSEN may not generate as many or as large a pore in the cell membrane as it increases cell membrane activity and permeability.

It is to be understood, however, that the LSEN meshes and electrodes and their combinations are structurally altered according to the present invention to be adapted for optimum use for each of the solid organs and tissues disclosed and claimed in the present application. For example, the LSEN meshes and electrodes and their combinations for use with the liver are specially arranged and configured for creating an LSEN field in the liver depending on whether the application is ex vivo, in vivo and where the latter, whether it is used inside or outside the body. Similarly, the shape and size of the LSEN meshes and electrodes and their combinations for use with the lung or portions thereof will be structurally altered to be optimal for that application as opposed to the shape and sized used with the liver. Further, it is to be understood that there is considerable individual variation in organ size and shape from one patient to another. Therefore, individualization of shape and size is to be expected, certainly between infant, juvenile and adult patients as well as having a design and construction which is customizable at the site of application by the surgeon. For example, a negative mesh of a universal size and shape can be constructed so that it is capable of being trimmed to size and shape for each individual application.

The list of molecules and their inhibitors, enhancers, regulator, genes, siRNAs, shRNAs, antigens, antibodies, and peptides that are related with these molecules, that can be used in the invention is extensive. A listing for the arthritis and other orthopedic diseases is set forth in U.S. Provisional Patent Application serial No. 60/894,877, filed on Mar. 14, 2007, and U.S. patent application Ser. No. ______ filed on ______, entitled, Method And Apparatus Of Low Strength Electric Field Network-Mediated Delivery Of Drug, Gene, Si-Rna, shRNA, Protein, Peptide, Antibody Or Other Biomedical And Therapeutic Molecules And Reagents In Solid Organs, which is incorporated herein by reference. The following list is to be understood as illustrative and not limiting with respect to the possible transfected materials using the invention.

a. Cytokines:
i. Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1.
ii. Other Cytokines: AREG, BMP1, BMP2, BMP3, BMP7, CAST, CD40LG, CER1, CKLFSF1, CKLFSF2, CLC, CSF1, CSF2, CSF3, CTF1, CXCL16, EBI3, ECGF1, EDA, EPO, ERBB2, ERBB21P, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, IL1B, IL1F10, IL1F5, IL1F6, IL1F7, IL1F8, IL1F9, IL1RN, IL2, IL20, IL21, IL22, IL23A, IL24, IL26, IL27, IL28B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN, SCGB1A1, SCGB3A1, SCYE1, SDCBP, SECTM1, SIVA, SLCO1A2, SLURP1, SOCS2, SPP1, SPRED1, SRGAP1, THPO, TNF, TNFRSF11B, TNFSF10, TNFSF11, TNFSF13, TNFSF13B, TNFSF14, TNFSF15, TNFSF18, TNFSF4, TNFSF7, TNFSF8, TNFSF9, TRAP1, VEGF, VEGFB, YARS.
b. Cytokine Receptors:
i. Cytokine Receptors: CNTFR, CSF2RA, CSF2RB, CSF3R, EBI3, EPOR, F3, GFRA1, GFRA2, GHR, IFNAR1, IFNAR2, IFNGR1, IFNIGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, ILIRL2, IL20RA, IL21R, IL22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN.
ii. Chemokine Receptors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1.
c. Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15.
d. Cytokine Production: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, INS, IRF4, NALP12, NFAM1, NOX5, PRG3, S100B, SAA2, SFTPD, SIGIRR, SPN, TLR1, TLR3, TLR4, TLR6, TNFRSF7.
e. Other Genes involved in Cytokine-Cytokine Receptor Interaction: ACVR1, ACVR1 B, ACVR2, ACVR2B, AMH, AMHR2, BMPR1A, BMPR1B, BMPR2, CCR1, CD40, CRLF2, CSF1R, CXCR3, IL18RAP, IL23R, LEP, TGFB1, TGFB2, TGFB3, TGFBR1, TGFBR2, TNFRSF1A, TNFRSF1B, TNFRSF21, TNFRSF8, TNFRSF9, XCR1.
f. Acute-Phase Response: AHSG, APCS, APOL2, CEBPB, CRP, F2, F8, FN1, IL22, IL6, INS, ITIH4, LBP, PAP, REG-III, SAA2, SAA3P, SAA4, SERPINA1, SERPINA3, SERPINF2, SIGIRR, STAT3.
g. Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, CD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, IL1F10, IL1F5, IL1F6, IL1R1, IL1RAP, IL1RN, IL20, IL22, IL31RA, IL5, IL8, IL8RA, IL8RB, IL9, IRAK2, IRF7, ITCH, ITGAL, ITGB2, KNG1, LTA4H, LTB4R, LY64, LY75, LY86, LY96, MEFV, MGLL, MIF, MMP25, MYD88, NALP12, NCR3, NFAM1, NFATC3, NFATC4, NFE2L1, NFKB1, NFRKB, NFX1, NMI, NOS2A, NR3C1, OLR1, PAP, PARP4, PLA2G2D, PLA2G7, PRDX5, PREX1, PRG2, PRG3, PROCR, PROK2, PTAFR, PTGS2, PTPRA, PTX3, REG-III, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1.
h. Humoral Immune Response: BATF, BCL2, BF, BLNK, C1 R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSF1R, CSF2RB, CXCR3, CYBB, EBI3, FADD, GPI, 110, IL12A, IL12B, IL12RB1, IL13, IL18, IL1B, IL2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1.
i. IL-1 R/TLR Members and Related Genes:
i. Detection of Pathogens: TLR1, TLR3, TLR4, TLR6, TLR8. Interleukin-1 Receptors: IL1R1, IL1R2, IL1RAP, IL1RAPL2, IL1RL2.
ii. Other Genes Involved in the IL-1R Pathway: IKBKB, MAPK14, MAPK8.
iii. Inflammatory Response: MA, IL1B, IL1F10, IL1F5, IL1F6, IL1F8, IL1R1, IL1RN, IRAK2, MYD88, NFKB1, TLR1, TLR10, TLR2, TLR3, TLR4, TLR6, TLR8, TLR9, TNF, TOLLIP.
iv. Apoptosis: IL1A, IL1B, NFKB1, NFKBIA, TGFB1, TNF.
v. Cytokines: IFNA1, IFNB1, IL1A, IL1B, IL1F10, IL1F5, IL1F6, IL1F7, IL1F8, IL1F9, IL6, TNF.
iv. Genes Involved in NFKB Signaling: CHUK, IRAK2, MYD88, TLR1, TLR3, TLR4, TLR6, TLR8, TRAF6.
j. Host Defense to Bacteria:
i. Detection of Bacteria: CD1D, PGLYRP1, PGLYRP2, PGLYRP3, TLR1, TLR3, TLR6.
ii. LSP Receptor: CD14, CXCR4, DAF.
iii. Acute-phase Response: CRP, FN1, LBP.
iv. Complement Activation: C5, C8A, DAF, PFC.
v. Inflammatory Response: AZU1, C5, CCL2, CD14, CRP, CYBB, LY96, NFKB1, NOS2A, PRG2, S100A12, STAB1, TLR1, TLR3, TLR6, TLR9.
vi. Cytokines, Chemokines, and their Receptors: C5, CCL2, CXCR4, IFNGR1, IFNGR2, IL12RB2, PPBP.
vii. Antibacterial Humoral Response: CLECSF12, COLEC12, CYBB, DEFA5, DEFA6, LY96, NFKB1.
viii. Defense Response to Bacteria: AZU1, BPI, CAMP, CLECSF12, DCD, DEFA4, DEFA5, DEFA6, DEFB1, DEFB118, DEFB127, DEFB4, GNLY, HAMP, LALBA, LBP, LEAP-2, LTF, LYZ, NOS2A, PFC, PGLYRP1, PGLYRP2, PGLYRP3, PPBP, PRG2, RNASE3, RNASE7, S100A12, STAB1, TLR3, TLR6, TLR9.
ix. Other Genes Involved in the Host Defense Against Bacteria: CARD12, CHIT1, DMBT1, HAT, IRF1, NCF4, NFKBIA, PLUNC, SLC11A1.
k. Innate Immune Response:
i. Innate Immune Response: APOBEC3G, COLEC12, CRISP3, DEFB1, DEFB118, DEFB127, DMBT1, PGLYRP1, PGLYRP2, PGLYRP3, PLUNC, RNASE7, SFTPD, TLR8.
ii. Other Genes Involved in the Innate Immune Response: ARTS-1, CDID, IFNB1, IFNK, KIR3DL1, TLR10.
l. Septic Shock:
i. Apoptosis: ADORA2A, CASP1, CASP4, IL10, IL1B, NFKB1, PROC, TNF, TNFRSF1A.
ii. Cytokines and Growth Factors: CSF3, IL10, IL1B, IL6, MIF, TNF.
iii. Inflammatory Response: ADORA2A, CCR3, IL10, IL1B, IL1RN, MIF, NFKB1, PTAFR, TLR2, TLR4, TNF.
iv. Other Genes Involved in Septic Shock: GPR44, HMOX1, IRAK1, NFKB2, SERPINA1, SERPINE1, TREM1.
m. B-cell activation:
i. Antigen dependent B-cell activation: CD28, CD4, CD80, HLA-DRA, IL10, IL2, IL4, TNFRSF5, TNFRSF6, TNFSF5, TNFSF6.
ii. Other genes involved in B-cell activation: BLR1, HDAC4, HDAC5, HDAC7A, HDAC9, ICOSL, IGBP1, MS4A1, RGS1, SLA2.
iii. B-cell proliferation: CD81, IFNB1, IL10, TNFRSF5, TNFRSF7, TNFSF5.
iv. B-cell differentiation: AICDA, BLNK, GALNAC4S-6ST, HDAC4, HDAC5, HDAC7A, HDAC9, IL10, IL11, IL4, INHA, 1NHBA, KLF6, TNFRSF7.
n. B-cell activation:
i. Regulators of T-cell activation: CD2, CD3D, CD3E, CD3G, CD4, CD7, CD80, CD86, CD8A, CD8B1, CLECSF12, ICOSL, IRF4, KIF13B, NCK1, NCK2, PRLR, SIT, SLA2, TNFSF14.
ii. T-cell proliferation: CD28, CD3E, GLMN, ICOSL, IL10, IL12B, IL18, IL27, NCK1, NCK2, SFTPD, SPP1, TNFSF14.
iii. T-cell differentiation: CD1D, CD2, CD4, CD80, CD86, 1112B, IL2, IL27, IRF4, JAG2, NOS2A, RHOH, SOCS5, TNFRSF7, WWP1.
iv. Regulators of Th1 and Th2 development: ANPEP, CD2, CD33, CD5, CD7, CSF2, IFNA2, IFNB1, IFNG, IL10, IL12A, IL13, IL3, IL4, IL5, ITGAX, TLR2, TLR4, TLR7, TLR9, TNFRSF5.
v. Genes involved in Th1/Th2 differentiation: CD28, CD86, HLA-DRA, IFNG, IFNGR1, IFNGR2, IL12A, IL12B, IL12RB1, IL12RB2, IL18, IL18R1, IL2, IL2RA, IL4, IL4R, PVRL1, TNFRSF5, TNFSF5.
vi. Genes involved in T-cell polarization: CCL3, CCL4, CCR1, CCR2, CCR3, CCR4, CCR5, CCR7, CD28, CD4, CSF2, CXCR3, CXCR4, IFNG, IFNGR1, IFNGR2, IL12A, IL12RB1, IL12RB2, IL18R1, IL2, IL4, IL4R, IL5, TGFB1, TNFSF5.
o. Other genes related to immune cell activation:
i. Macrophage activation: C1QR1, IL31RA, INHA, INHBA, TLR1, TLR4, TLR6.
ii. Neutrophil activation: APOA2, IL8, PREX1, PRG3.
iii. Natural killer cell activation: CD2, IFNB1, IFNK, IL12B, IL2, IL21R, KIR3DL1, ULBP1, ULBP2, ULBP3.
iv. Others: AZU1, CX3CL1, ITIH1, TOLLIP, TXNDC, ZNF3.
p. B-cell activation:
i. Antigen dependent B-cell activation: CD28, CD4, CD80, HLA-DRA, IL10, IL2, IL4, TNFRSF5, TNFRSF6, TNFSF5, TNFSF6.
q. Other genes involved in B-cell activation: BLR1, HDAC4, HDAC5, HDAC7A, HDAC9, ICOSL, IGBP1, MS4A1, RGS1, SLA2.
r. B-cell proliferation: CD81, IFNB1, IL10, TNFRSF5, TNFRSF7, TNFSF5.
i. B-cell differentiation: AICDA, BLNK, GALNAC4S-6ST, HDAC4, HDAC5, HDAC7A, HDAC9, IL10, IL11, IL4, INHA, INHBA, KLF6, TNFRSF7.
s. T-cell activation:
i. Regulators of T-cell activation: CD2, CD3D, CD3E, CD3G, CD4, CD7, CD80, CD86, CD8A, CD8B1, CLECSF12, ICOSL, IRF4, KIF13B, NCK1, NCK2, PRLR, SIT, SLA2, TNFSF14.
t. T-cell proliferation: CD28, CD3E, GLMN, ICOSL, IL10, IL12B, IL18, IL27, NCK1, NCK2, SFTPD, SPP1, TNFSF14.
u. T-cell differentiation: CD1D, CD2, CD4, CD80, CD86, IL12B, IL2, IL27, IRF4, JAG2, NOS2A, RHOH, SOCS5, TNFRSF7, WWP1.
v. Regulators of Th1 and Th2 development: ANPEP, CD2, CD33, CD5, CD7, CSF2, IFNA2, IFNB1, IFNG, IL10, IL12A, IL13, IL3, IL4, IL5, ITGAX, TLR2, TLR4, TLR7, TLR9, TNFRSF5.
w. Genes involved in Th1/Th2 differentiation: CD28, CD86, HLA-DRA, IFNG, IFNGR1, IFNGR2, IL12A, IL12B, IL12RB1, IL12RB2, IL18, IL18R1, IL2, IL2RA, IL4, IL4R, PVRL1, TNFRSF5, TNFSF5.
x. Genes involved in T-cell polarization: CCL3, CCL4, CCR1, CCR2, CCR3, CCR4, CCR5, CCR7, CD28, CD4, CSF2, CXCR3, CXCR4, IFNG, IFNGR1, IFNGR2, IL12A, IL12RB1, IL12RB2, IL18R1, IL2, IL4, IL4R, IL5, TGFB1, TNFSF5. Y.
y. Other genes related to immune cell activation:
i. Macrophage activation: C1QR1, IL31RA, INHA, INHBA, TLR1, TLR4, TLR6.
ii. Neutrophil activation: APOA2, IL8, PREX1, PRG3.
iii. Natural killer cell activation: CD2, IFNB1, IFNK, IL12B, IL2, IL21R, KIR3DL1, ULBP1, ULBP2, ULBP3.
iv. Others: AZU1, CX3CL1, ITIH1, TOLLIP, TXNDC, ZNF3.
z. CTGFβ Superfamily Cytokines:
i. TGF-β: TGFB1, TGFB2, TGFB3. BMP: BMP1, BMP2, BMP3, BMP4, BMP5, BMP6, BMP7, BMP8B, BMP10, BMP15. GDF: AMH, GDF1, GDF2 (BMP9), GDF3 (Vgr-2), GDF5 (CDMP-1), GDF6, GDF7, GDF8, GDF9, GDF10, GDF11 (BMP11), GDF15, IGF1, IGFBP3, IL6, INHA (inhibin a), INHBA (inhibin BA), IVL (involucrin), LEFTY1, LEFTY2, LTBP1, LTBP2, LTBP4, NODAL, PDGFB, TDGF1.
ii. Activin: INHA (inhibin a), INHBA (inhibin BA), INHBB (inhibin BB), INHBC (inhibin BC), INHBE, LEFTY1, LEFTY2, NODAL.
aa. Receptors: ACVR1 (ALK2), ACVR1 B (ALK4), ACVR1C, ACVR2, ACVR2B, ACVRL1 (ALK1), AMHR2, BMPR1A (ALK3), BMPR1B (ALK6), BMPR2, ITGB5 (integrin B5), ITGB7 (integrin B7), LTBP1, MAP3K71P1, NROB1, STAT1, TGFB1I1, TGFBR1 (ALK5), TGFBR2, TGFBR3, TGFBRAP1.
bb. SMAD: SMAD1 (MADH1), SMAD2 (MADH2), SMAD3 (MADH3), SMAD4 (MADH4), SMAD5 (MADH5), SMAD6 (MADH6), SMAD7 (MADH7), SMAD9 (MADH9).
cc. SMAD Target Genes:
i. TGF-β Activin-responsive: CDC25A, CDKN1A (p21WAF1/p21CIP1), CDKN2B (p15LNK2B), COL1A1, COL1A2, COL3A1, FOS, GSC (goosecoid), IGF1, IGFBP3, IL6, ITGB5 (integrin B5), ITGB7 (integrin B7), IVL (involucrin), JUN, JUNB, MYC, PDGFB, SERPINE 1 (PAI-1), TGFB1I1, TGFB1I4, TGFB1, TGIF, TIMP1.
ii. BMP-Responsive: BGLAP (osteocalcin), DLX2, ID1, ID2, ID3, ID4, JUNB, SMAD6 (MADH6), SOX4, STAT1, TCF8.
iii. Molecules Regulating Signaling of the TGF-β Superfamily: BAMBI, BMPER, CDKN2B (p15LNK2B), CER1 (cerberus), CHRD (chordin), CST3, ENG (Evi-1), EVI1, FKBPIB, FST (follistatin), GREM1, HIPK2, MAP3K7, NBL1 (DAN), NOG, PLAU (uPA), RUNX1 (AML1), RUNX2, SMURF1, SMURF2, TDGF1.
dd. Adhesion and Extracellular Molecules:
i. < > BGLAP (osteocalcin), ENG (Evi-1), ITGB5 (integrin B5), ITGB7 (integrin B7), TGFB1I1, TGFBI.
ii. Extracellular Matrix Structural Constituents: BGLAP (osteocalcin), COL1A1, COL1A2, COL3A1, IVL (involucrin), LTBP1, LTBP2, LTBP4, TGFB1, TIMP1.
iii. Other Extracellular Molecules: AMH, BMP1, BMP10, BMP15, BMP2, FST (follistatin), GDF1, GDF10, GDF15, GDF2 (BMP9), GDF3 (Vgr-2), GDF9, GREM1, IGF1, IGFBP3, IL6, INHA (inhibin a), INHBA (inhibin BA), INHBB (inhibin BB), INHBC (inhibin BC), PDGFB, PLAU (uPA), SERPINE1.
   ee. Transcription Factors and Regulators: DLX2, EVI1, FOS, GSC (goosecoid), HIPK2, ID1, ID3, ID4, JUN, JUNB, MYC, NROB1, RUNX1 (AML1), RUNX2, SMAD1 (MADH1), SMAD2 (MADH2), SMAD3 (MADH3), SMAD4 (MADH4), SMAD5 (MADH5), SMAD6 (MADH6), SMAD7 (MADH7), SMAD9 (MADH9), SMURF2, SOX4, STAT1, TCF8 (AREB6), TGFB1I1, TGFB1I4, TGIF.
   ff. Genes Involved in Cellular and Developmental Processes:
   i. Apoptosis: CDKN1A (p21WAF1/p21CIP1), HIPK2, IGFBP3, INHA (inhibin a), INHBA (inhibin BA), STAT1, TDGF1, TGFB1.
   ii. Embryonic Development: BMP10, BMP4, GDF11 (BMP11), INHBA (inhibin BA), SMAD3, SMURF1, TDGF1.
   iii. Muscle Development: GDF8, GDF9, IGF1, SMAD3. Neurogenesis: DLX2, GDF11 (BMP11), GREM1, INHA (inhibin a), INHBA (inhibin BA), NOG.
   iv. Reproduction: AMH, AMHR2, BMP15, FST (follistatin), GDF9, INHA (inhibin a), INHBA (inhibin BA), INHBB (inhibin BB), INHBC (inhibin BC), LEFTY2, NROB1, TDGF1.
   v. Skeletal Development: BGLAP (osteocalcin), BMP1, BMP2, BMP3, BMP4, BMP5, BMP6, BMP7, BMP8B, BMPR2, CHRD (chordin), COL1A1, COL1A2, GDF10, GDF11 (BMP11), IGF1, INHA (inhibin a), INHBA (inhibin BA), NOG, RUNX2.
   gg. TH1 Cytokines and Related Genes: CCR5, CD28, CSF2 (GM-CSF), CXCR3, HAVCR2 (TIM3), IFNG, IGSF6 (CD40L), IL12B, IL12RB2, IL18, IL18BP, IL18R1, IL2, IL2RA (CD25), IRF1, SOCS1 (SSI-1), SOCS5, STAT1, STAT4, TBX21 (T-bet), TNF.
   hh. TH2 Cytokines and Related Genes: CCL11 (eotaxin), CCL15 (MIP-1d), CCL5 (RANTES), CCL7 (MCP-3), CCR2 (MCP-1), CCR3, CCR4, CCR9, CEBPB, FLJ14639 (NIP45), GATA3, GFI1, GPR44 (CRTH2), ICOS, IL10, IL13, IL13RA1, IL13RA2, IL1R1, IL1R2, IL4, IL4R, IL5, IL9, IRF4, JAK1, JAK3, MAF, NFATC1 (NFATc), NFATC2 (NFATp), NFATC3 (NFAT4), NFATC4, RNF110 (ZNF144), STAT6, TLR4, TLR6, TMED1, ZFPM2 (FOG2).
   ii. CD4+ T Cell Markers: BCL3 (p50), CD4, CD69, CD80, CD86, CREBBP (CBP), CTLA4, IL15, IL6, IL6R, IL7, JAK2, LAGS, LAT, MAP2K7 (JNKK2), MAPK10 (JNK-3), MAPK8 (JNK-1), MAPK9 (JNK-2), PTPRC (CD45), SOCS3 (SSI-3), TFCP2 (CP2), TGFB3, TNFRSF21 (DR6), TNFRSF7 (CD27), TNFRSF8 (CD30), TNFRSF9 (4-1 BB), TNFSF4 (OX-40), TNFSF5 (CD40), TNFSF6 (FasL), TYK2, YY1.
   jj. Immune Cell Activation:
   i. T-cell Activation: CD2, CD28, CD4, CD80, CD86, GLMN, IL10, IL12B, IL18, IL2, IL27, IRF4, SFTPD, SOCS5, SPP1, TNFRSF7 (CD27).
   ii. B-cell Activation: IL10, IL4, INHA, INHBA, TNFRSF7 (CD27), TNFSF5 (CD40).
   kk. T-helper 1 Type Immune Response: CD4, CD80, CD86, GLMN, 110, IL17F, IL18, IL18BP, INHA, INHBA, IRF4, SFTPD, SPP1, TLR4, TLR6, IL12B, IL27, TNFRSF7 (CD27).
   ll. T-helper 2 type Immune Response: CD86, IL10, IL18, IL4, IRF4.
   mm. Antimicrobial Humoral Response: CCL15 (MIP-1d), CCL7 (MCP-3), CCR2 (MCP-1), CXCR3, FADD (Fas), IL12B, IL13, NFKB1, SFTPD, YY1.
   nn. Other Immune Response Genes: CSF2 (GM-CSF), FOSL1 (Fra-1), CEBPB, FOS, IRF1, MHC2TA (CIITA), SOCS6.
   oo. Transcription Factors and Regulators:
   i. Positive Regulation of Transcription: CD80, CD86, IRF4.
   ii. RNA Polymerase II Transcription Factor Activity: ATF2, FOS, GFI1, IRF4, JUN, JUNB, JUND, MAF, MHC2TA (CIITA).
   iii. Transcription Co-activator Activity: ATF2, CREBBP (CBP), JUNB, MHC2TA (CIITA), NFATC3, NFATC4, YY1.
   iv. Transcription Co-repressor Activity: JUNB, YY1.
   v. Transcription Factor Activity: CEBPB, CREBBP (CBP), FOSL1 (Fra-1), FOSL2 (Fra-2), GATA3, IRF1, JUND, NFATC1 (NFATc), NFATC2 (NFATp), NFATC3 (NFAT4), NFATC4, NFKB1, RNF110 (ZNF144), STAT1, STAT4, STATE, TBX21 (T-bet), TFCP2 (CP2), YY1.
   vi. Transcription from Pol II Promoter: CEBPB, FOSL1 (Fra-1), GATA3, IRF1, MAF, NFATC1, NFATC3, NFATC4, NFKB1, STAT1.
   vii. Other Transcription Factors and Regulators: BCL3 (p50), JUN (c-JUN), SOCS2 (STATI2), SOCS4 (CIS4), SOCS6, SOCS7 (SOCS4), TH1L, TNF, ZFPM2 (FOG2).
   pp. Toll-Like Receptors: LY64 (RP105/CD180), SIGIRR (TIR8), TLR1, TLR2, TLR3, TLR4, TLR5, TLR6, TLR7, TLR8, TLR9, TLR10.
   qq. Adaptors & TLR Interacting Proteins: BTK, CD14, GPC1 (SP-A), HMGB1, HRAS, HSPA1A, HSPA4, HSPA6, HSPD1, LY86 (MD-1), LY96, MAL, MAPK81P3 (JIP3), MYD88, PELI1 (Pellino 1), PELI2 (Pellino 2), PGLYRP1, PGLYRP2, PGLYRP3, PGLYRPIbeta, RIPK2 (RIP2), SARM1, TICAM2, TIRAP, TOLLIP, TRIF (TICAM1).
   rr. Effectors: CASP8, EIF2AK2, FADD, IRAK1, IRAK2, IRAK3, IRAK4, MAP3K7, MAP3K71P1 (TAB1), MAP3K71P2 (TAB2), NR2C2 (TAK1), PPARA, PRKRA (PKR), SITPEC (ECSIT), TRAF6, UBE2N (Ubc13), UBE2V1 (Uev1A).
   ss. Downstream Pathways and Target Genes:
   i. NFKB Pathway: CCL2 (MCP-1), CHUK (IKK-a), CSF2 (GM-CSF), CSF3 (G-CSF), IFNB1, IFNG, IKBKB (IKK-b), IKBKG (IKK-g), IL1A, IL1B, IL2, IL6, IL8, IL10, IL12A, IL12B, LTA (TNF-b), MAP3K1 (MEKK1), MAP3K14, MAP4K4 (NIK), NFKB1, NFKB2, NFKBIA (IkBa/mad3), NFKBIB (IkBb), NFKBIE, NFKBIL1, NFKBIL2, NFRKB, REL, RELA, RELB, TNF (TNFa), TNFRSF1A, TRADD.
   ii. IL JNK/p38 Pathway: ELK1, FOS, JUN, MAP2K3 (MKK3), MAP2K4 (MKK4), MAP2K6 (MKK6), MAP3K1 (MEKK1), MAPK8 (JNK1), MAPK9 (JNK2), MAPK10, MAPK11 (p38bMAPK), MAPK12 (p38gMAPK), MAPK13, MAPK14 (p38 MAPK).
   iii. NF/IL6 Pathway: CLECSF9, PTGES, PTGS2 (Cox-2).
   iv. IRE Pathway: CXCL10 (IP-10), IFNB1, IFNG, IRF1, IRF3, IRF7, TBK1.
   tt. Regulation of Adaptive Immunity: CD80, CD86, RIPK2 (RIP2), TRAF6.
   uu. Growth factor and associated molecule: BMP1, BMP2, BMP3, BMP4, BMP5, BMP6, BMP7, BMP8, BMPR1A, CASR, CSF2 (GM-CSF), CSF3 (G-CSF), EGF, EGFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKB1, PDGFA, RUNX2 (CBFA1), SOX9, TGFB1, TGFB2, TGFB3, TGFBR1, TGFBR2, TNF (TNFa), TWIST, VDR, VEGF, VEGFB, VEGFC
   vv. Matrix and its associated protein: ALPL, ANXA5, ARSE, BGLAP (osteocalcin), BGN, CD36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, COL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin)
   ww. Cell adhesion molecule: ICAM1, ITGA1, ITGA2, ITGA3, ITGAM, ITGAV, ITGB1, VCAM1
   xx. Cell Growth and Differentiation:
   i. Regulation of the Cell Cycle: EGFR, FGF1, FGF2, FGF3, IGF1R, IGF2, PDGFA, TGFB1, TGFB2, TGFB3, VEGF, VEGFB, VEGFC.
   ii. Cell Proliferation: COL18A1, COL4A3, CSF3, EGF, EGFR, FGF1, FGF2, FGF3, FLT1, IGF1, IGF1R, IGF2, PDGFA, SMAD3, SPP1, TGFB1, TGFB2, TGFB3, TGFBR2, VEGF, VEGFB, VEGFC.
   iii. Growth Factors and Receptors: BMP1, BMP2, BMP3, BMP4, BMP5, BMP6, BMP7, BMP8B, BMPR1A, CSF2, CSF3, EGF, EGFR, FGF1, FGF2, FGF3, FGFR1, FGFR2, FGFR3, FLT1, GDF10, IGF1, IGF1R, IGF2, PDGFA, SPP1, TGFB1, TGFB2, TGFB3, TGFBR1, TGFBR2, VEGF, VEGFB, VEGFC.
   iv. Cell Differentiation: SPP1, TFIP11, TWIST1, TWIST2.
   yy. Extracellular Matrix (ECM) Molecules:
   i. Basement Membrane Constituents: COL4A3, COL4A4, COL4A5, COL7A1, SPARC.
   ii. Collagens: COL10A1, COL11A1, COL12A1, COL14A1, COL15A1, COL16A1, COL18A1, COL19A1, COL1A1, COL1A2, COL2A1, COL3A1, COL4A3, COL4A4, COL4A5, COL5A1, COL7A1, COL9A2.
   iii. ECM Protease Inhibitors: AHSG, COL4A3, COL7A1, SERPINH1.
   iv. ECM Proteases: BMP1, CTSK, MMP10, MMP13, MMP2, MMP8, MMP9, PHEX.
   v. Structural Constituents of Bone: BGLAP, COL1A1, COL1A2, MGP.
   vi. Structural Constituents of Tooth Enamel: AMBN, AMELY, ENAM, STATH, TUFT1.
   vii. Other ECM Molecules: BGN, BMP2, BMP8B, COL17A1, COMP, CSF2, CSF3, DCN, DSPP, EGF, FGF1, FGF2, FGF3, FLT1, GDF10, IBSP, IGF1, IGF2, PDGFA, SPP1, VEGF, VEGFB.
   zz. Cell Adhesion Molecules:
   i. Cell-cell Adhesion: CDH11, COL11A1, COL14A1, COL19A1, ICAM1, ITGB1, VCAM1.
   ii. Cell-matrix Adhesion: ITGA1, ITGA2, ITGA3, ITGAM, ITGAV, ITGB1, SPP1.
   iii. Other Cell Adhesion Molecules: BGLAP, CD36, COL12A1, COL15A1, COL16A1, COL18A1, COL4A3, COL5A1, COL7A1, COMP, FN1, IBSP, SCARB1, TNF.
   aaa. Transcription Factors and Regulators: MSX1, MSX2, NFKB1, RUNX2, SMAD1, SMAD2, SMAD3, SMAD4, SMAD5, SMAD6, SMAD7, SMAD9, SOX9, TNF, TWIST1, TWIST2, VDR.

Using a previously developed rabbit heterotopic functional heart transplant model, we have found that the efficiency of electric field network-mediated ex vivo intracoronary interleukin-10 (IL-10) gene transfer in heart was higher than that carried by adenovirus. Localized transgene expression was initiated earlier and lasted longer. The transgene and its protein were more homogeneously distributed. No ectopic transfection, cellular toxicity, autoimmunogenecity, arrhythmogenic and other cardiac adverse effects were found. Most importantly we found this approach has the potential to transfer multiple genes simultaneously.

Previously, several candidate genes have been reported that could prolong allograft survival in non-functional heterotopic heart transplant model of rodent that includes IL-10, CTLA4Ig, CD40Ig and TGF-β. Adenovirus-mediated combination of CTLA4Ig and CD40Ig gene therapy has induced long-term acceptance of rat cardiac allografts. However, true tolerance was never achieved. We have demonstrated in the case of the illustrated embodiment long-term survival and substantial improvement in the function of rabbit cardiac allografts induced by liposome-mediated IL-4 and IL-10 combined gene therapy. In the illustrated embodiment we used a low strength electric field network (LSEN) system to transfer ex vivo a vector containing two cytokine genes, IL-4 and IL-10, and a positron emission tomograph (PET) reporter gene sr39TK in the same model. Fully, two thirds of the allografts survived indefinitely. Our preliminary results demonstrate even better outcomes of electric field-IL-10-CTLA4Ig gene therapy in this rat model. Most recently, we have used an electric field-mediated ex vivo to transfer a small interference RNA (siRNA) to target class II transactivator (CIITA), the master regulator of MHC class II gene expression.

Delivery of genes or macromolecules to cardiovascular tissues holds great promise for the treatment of many acquired and inherited diseases. The time interval between harvest and implantation of cardiac allografts is used to biologically modify the graft. Localized gene transfer introduces immunosuppressive molecules only into the graft, thereby limiting systemic side effects, and prolonging allograft survival. The illustrated embodiment successfully efficiently and safely transfers a gene or genes into the target cells for immunosuppression, and simultaneously successfully efficiently and safely transfers a proper candidate gene or genes for a particular disease. As we have demonstrated shown in provisional patent application cited above entitled, “Method And Apparatus Of Low Strength Electric Field Network-Mediated Delivery Of Drug, Gene, Si-RNA, Protein, Peptide, Antibody Or Other Biomedical And Therapeutic Molecules And Reagents In Solid Organs”, we developed a novel low strength (≦10 v/cm) electric field network (LSEN)-mediated gene transfer approach for ex vivo gene transfer in the whole heart of large animal and human. We have optimized the design and the pulse parameters for the ex vivo gene transfer using our rabbit heterotopic functional cervical cardiac isograft transplant model so that the efficiency of LSEN-mediated ex vivo intracoronary interleukin-10 (IL-10) gene transfer in heart was higher than that carried by adenovirus.

Subsequently, we used LSEN to ex vivo transfer a vector containing two cytokine genes, IL-4 and IL-10, and a positron emission tomograph (PET) reporter gene sr39TK in the same model resulted indefinite survival in two thirds of the allografts. Our most recent preliminary results demonstrate even better outcomes of electropermeabilization-IL-10-CTLA4Ig gene therapy in the same rat model. Our results suggest that ex vivo LSEN technique has a potential to transfer multiple genes simultaneously either in single vector or in multiple vectors.

Most recently, a LSEN-mediated ex vivo transferring a small interference RNA (siRNA) technique has been established for targeting the class II transactivator (CIITA), the master regulator of MHC class II gene expression. On the other hand, our study also demonstrates that the rabbit heterotopic functional heart transplant model is a useful tool for translational studies in developing clinically applicable gene therapy method for heart transplantation. However, this approach needs to be refined upon the defining the most effective candidate gene(s) that can induce T cell anergy and true tolerance, optimize the siRNA, transferring technique, establish the most accurate noninvasive PET transgene quantification method, and be characterized for its pharmacokinetics and pharmacodynamics for the prevention and treatment of heart transplant rejection.

Our study has shown that LSEN efficiently facilitates the localized transfer of multiple genes in large animal heart, without ectopic gene transfection and without significant cardiac adverse effect and autoimmunogenecity. This paves the way for localized combinatorial immunosuppressive gene therapy in heart transplantation. We have a powerful localized gene transfer method, which becomes even more powerful when combined with a candidate gene(s) that induces T cell anergy and true tolerance.

Previous studies have shown that costimulation blockage, CTLA4Ig or CD40Ig transfection, could prolong cardiac allograft survival. Over expressing exogenous immunosuppressive cytokine, IL-10 or TGF-β, could also prolong the cardiac survival. Combination of IL-10 and IL-4, or CTLAIg and CD40Ig significantly extents their immunosuppressive effect and induces allograft long-term survival. However, individually, none of them can induce true T cell anergy and tolerance. It is noteworthy that all of the prior gene therapy studies use an adenovirus, except IL-10 and IL-10 transfection studies which were nonviral. We found that using LSEN combined with IL-4 and IL-10 gene therapy induces indefinite survival in ⅔ of allografts, but IL-4 induced excessive effusion and seroma formation surrounding allografts in the recipient rabbits' neck in an early stage and caused compression on the allografts and led to 17% allograft failure. The combination of IL-10 and CTLA4Ig produced the best outcome with 78% allografts indefinitely surviving, but true T cell anergy and tolerance are still not induced.

The complex immune mechanisms that lead to full T cell activation and allograft rejection requires two distinct signals. The first signal originates with the engagement of an allogenic major histocompatibility complex (MHC) antigen when it complexes with the receptor on the recipient's T cell membrane. Recent studies in a knock-out and transgenic mice model showed that allografts lacking MHC II had prolonged survival, but not in MHC I-deficient allografts. The second signal required is provided by engagement of one or more T cell surface receptors with their ligands on antigen presenting cells (APC). Cardiac myocytes of the allograft also served as the APC in this circumstance. Among the multiple costimulatory pathways identified, two types of costimulatory interactions are critical for antigen-specific T cell activation in the development of productive immunity, namely CD28-B7 and CD4O-CD40L (CD154) interactions. To induce full antigen-specific T cell unresponsiveness, we have to block both signals.

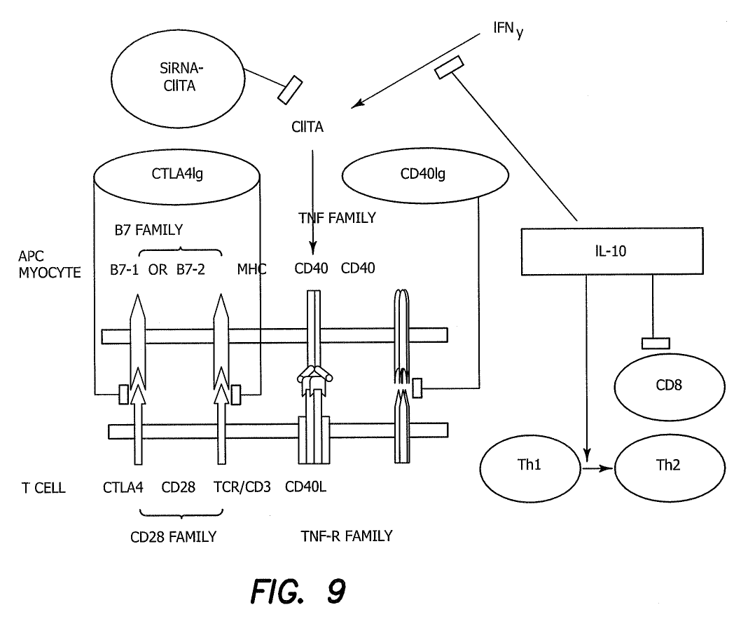

Our most recent study has demonstrated that a triple-immunosuppression strategy may effectively induce T cell anergy. We simultaneously transfer CTLA4Ig and CD40Ig to block two major co-stimulatory pathways, and CIITA-siRNA to down regulate MCHII expression in the cardiac allograft. As diagrammatically depicted in FIG. 9 CIITA is the most important transcription factor for the regulation of genes required for MHCII-restricted antigen-presentation. Expression of classical and non-classical MHCII is mainly at the level of transcription and regulated primarily by CIITA. Fetal trophoblasts lack expression of MHCII molecules due to the lack of CIITA expression, both constitutively and after exposure to IFNy. The absence of MHCII molecules on trophoblasts is thought to play a critical role in preventing rejection of the fetus by the maternal immune system. Mice CIITA−/− knock-out cardiac allografts survive four times longer than that the controls. Thus, using small interference RNA (siRNA) to knock down the expression of CIITA is the ideal strategy for blocking MHCII activation. In combination with the blockage of two co-stimulatory pathways using immunoglobulin fusion proteins, CTLA4Ig and CD40Ig alloantigen-specific T cell unresponsiveness may be induced.

Turn now to the illustrated embodiment of the method and its proof of concept. Below we first describe results of optimization of LSEN electric field parameters, then present our data on validating the feasibility of low strength electric field network (LSEN)-mediated ex vivo gene transfer in rabbit heart, then present our preliminary data on the efficiency and efficacy of low strength electric field network-mediated multiple immunosuppressive gene transfer in cardiac allografts. We use a highly efficient and safe low strength electric field network-mediated ex vivo gene transfer technique for ex vivo gene transfer in large animal and human hearts. We further validated this technique using a clinically relevant heterotopic function heart transplant rabbit model.

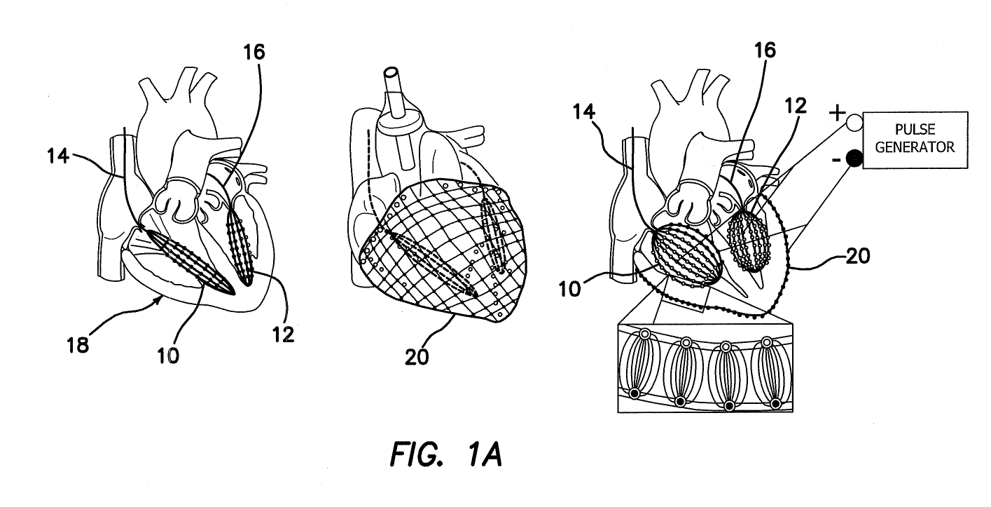

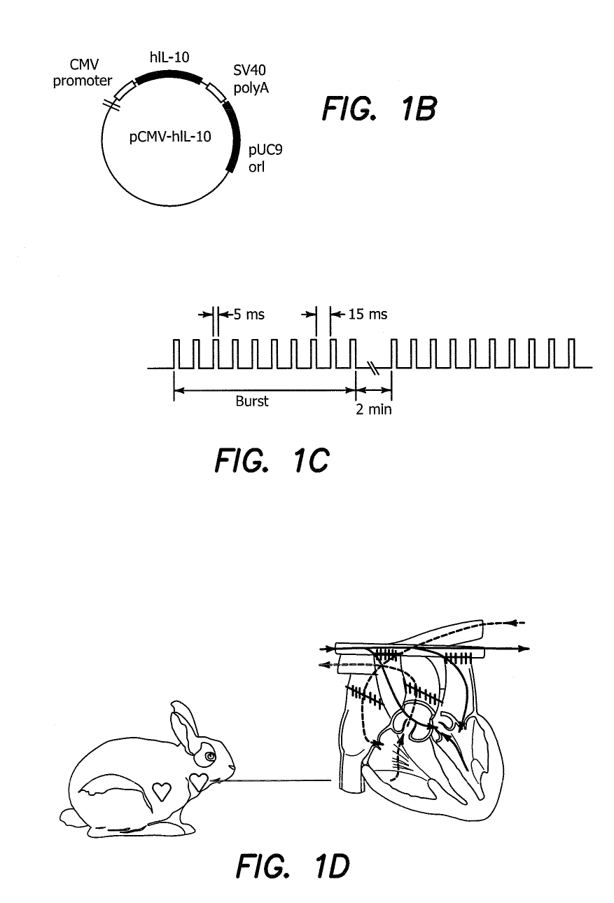

To achieve uniform electropermeabilization in an entire organ ex vivo, we designed a set of electrode arrays that are comprised of a pair of dense electrode arrays or “baskets” 10, 12 on corresponding catheters 14, 16 that are inserted inside the heart 18 as depicted in the cross sectional view of the left most portion of FIG. 1a. The unfurled “baskets” 10, 12 are deployed in two ventricles allow all LSEN electrodes to directly contact the endomyocardium, and a dense LSEN electrode array or “mesh” 20 of opposite polarity is fitted over the exterior surface of the heart 18 as shown in the centermost plan view of FIG. 1a.

The distances between the closest adjacent electrodes on electrode arrays 10, 12 when deployed or expanded inside the heart 18 and the electrode array 20 on the outside of the heart 18 are minimized to approximately be only the thickness of the heart wall itself as shown in the rightmost portion of FIG. 1a and the inset showing a portion of a ventricular wall in enlarged scale. The voltage applied to the interior electrode arrays 10, 12 and exterior electrode array 20 provides a dense electric field fringe network for electropermeabilizing the whole heart according to the LSEN methodology disclosed in the incorporated applications above. Intravascular gene delivery during and after application of the field allows continuous perfusion of the gene-carrying medium to virtually every cell in the heart and is an essential step. Theoretically, performing uniform electropermeabilization and intravascular gene delivery simultaneously in the heart 18 will result in a homogeneous transgene expression in every cell of a whole organ.

We validated the concept and feasibility of low strength electric field network-mediated plasmid-human interleukine-10 gene (phIL-10) diagrammatically depicted in FIG. 1b transfer using the LSEN signal of FIG. 1c and using a rabbit whole heart ex vivo intracoronary gene delivery and heterotopic functional cervical heart transplantation model we established previously and which is diagrammatically illustrated in FIG. 1d. The rabbit heterotopic functional heart transplant model is a useful tool to systematically validate and compare the testing the efficiency, efficacy and adverse effects of adenovirus-mediated and liposome-mediated ex vivo intracoronary gene transfer previously.

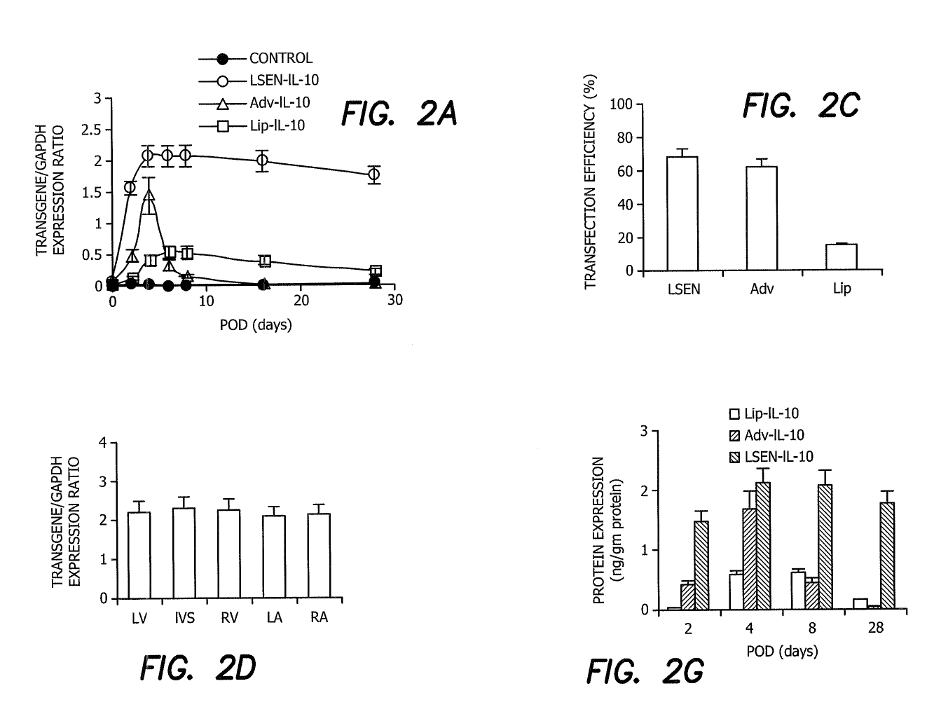

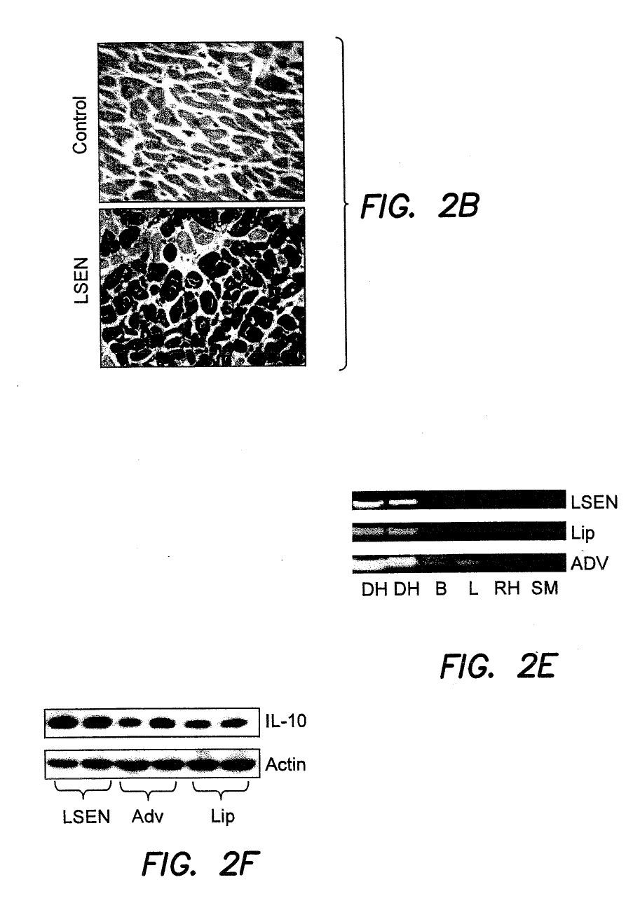

Using a pair of primers specifically for hIL-10 gene, we examined the magnitude, time-course and the distribution of LSEN-mediated ex vivo gene transfer induced transgene expression in the donor hearts 18. LSEN-mediated gene transfer induced hIL-10 transgene over expression was initiated within 3 hours, rapidly reached the peak in 2 to 3 days then slowly declined thereafter as shown in the graph of FIG. 2a. FIG. 2a shows that the increase of hIL-10 mRNA level was initiated significantly earlier and the magnitude of the transgene expression was significantly higher than that induced by adenovirus- or liposome-mediated hiL-10 gene transfer in the same animal model. In contrast to the transient transfection induced by adenovirus, the transgene over expression induced by LSEN-phIL-10 remained much longer than that by liposome. The efficiency of LSEN-mediated gene transfer was 1360 times higher than that of phIL-10 only, 4.5 times higher than that of liposome-phIL-10, and even significantly higher than that of adenovirus-phIL-10 as showing the histological microphotographs of FIG. 2b and the corresponding bar chart of FIG. 2c.

LSEN-phIL-10 induced transgene expression was homogeneously distributed in whole donor hearts. Like in adenovirus-phIL-10 and liposome-phIL-10 treated donor hearts as depicted in the bar chart of FIG. 2d, a relatively higher transgene expression was also observed in the vessel wall than cardiac myocytes in LSEN-phIL-10 group. Most importantly, the transgene expression was localized only in the targeted donor heart, but not observed in the recipient rabbits' native hearts, or any other organs and tissues as depicted in the gel data graphs of FIG. 2e. In contrast, ectopic transgene expression was observed in all recipient rabbits in adenovirus-phIL-10 group. LSEN-mediated gene transfer induced a homogeneous IL-10 protein over expression in the whole donor hearts as depicted in the data graphs of FIG. 2f.

The time-course of IL-10 protein expression was parallel with the transgene expression as shown in the bar chart of FIG. 2g. The maximum IL-10 protein over expression in the left ventricular myocardium of LSEN-phIL-10 group was 3.3 times higher than that in liposome-phIL-10 group, and significantly higher than that in adenovirus-phIL-10 group. At POD 28, the IL-10 protein expression level was 20 times higher that in adenovirus-phIL-10 group.

In summary, these findings constitute the first proof-of-principle study for the feasibility of low strength electric field network (LSEN) induced ex vivo intracoronary delivered gene transfer in large animal hearts 18, and demonstrate the feasibility of this strategy for gene therapy in heart transplantation.

Consider now the optimization of low strength electric field network-mediated ex vivo IL-10 gene transfer in hearts. Besides gene concentration, medium and intracoronary gene delivery that we have previously optimized for adenovirus- and liposome-mediated gene transfer, consider the electric conditions such as electrical pulse strength, length, interval and number of pulses must be tested for the optimization of LSEN efficiency. We used the same heterotopic functional cervical heart transplant rabbit model as described above. The optimal parameters were: pulse length (5 ms), pulse interval (15 ms), number of pulse (10), number of burst (10), burst-interval (2 min), defined using an isolated rabbit heart tissue gene transfer model as a standard set of parameters while we test each variable parameter in rabbit heart transplant model.

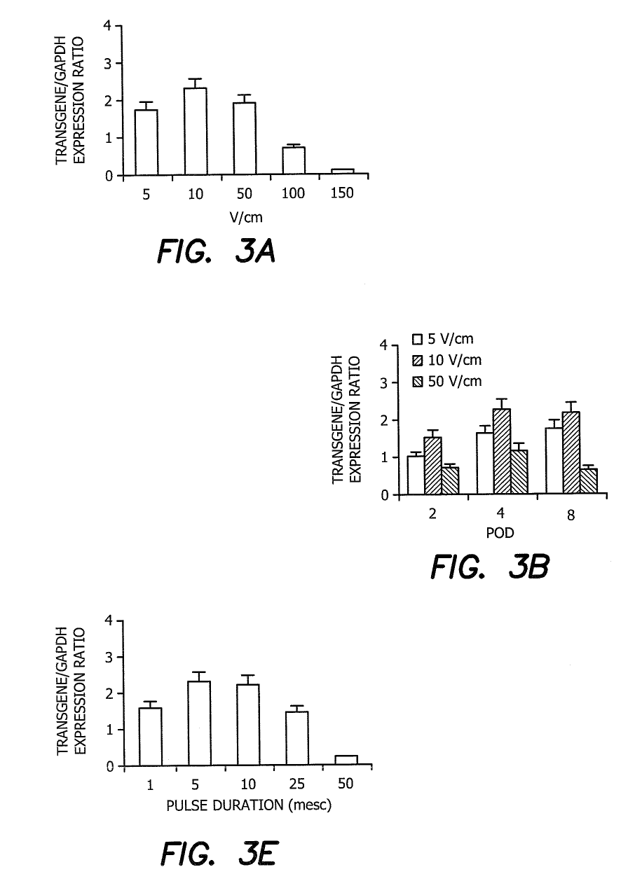

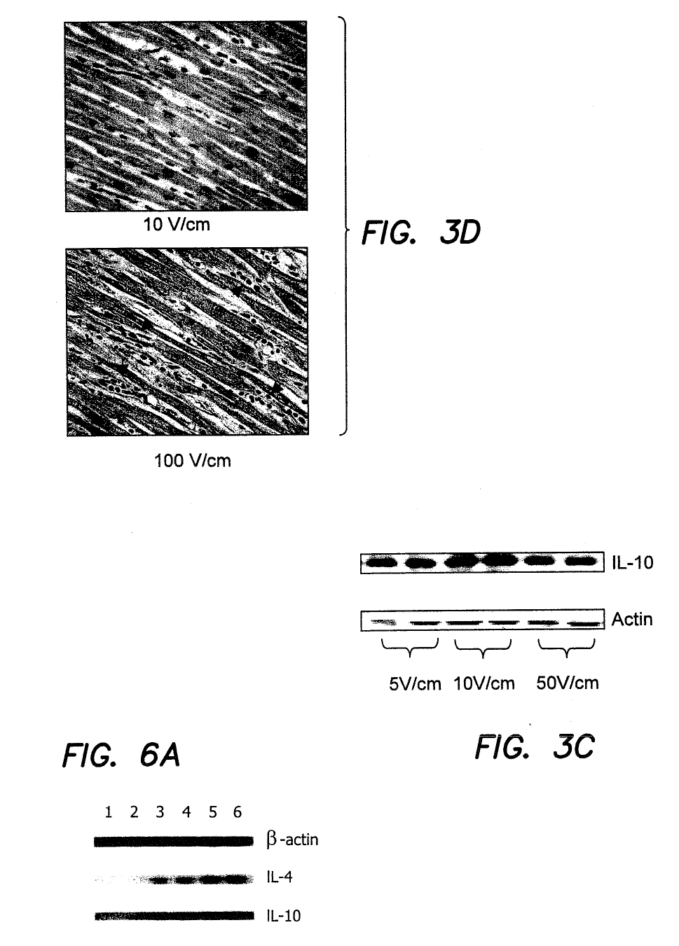

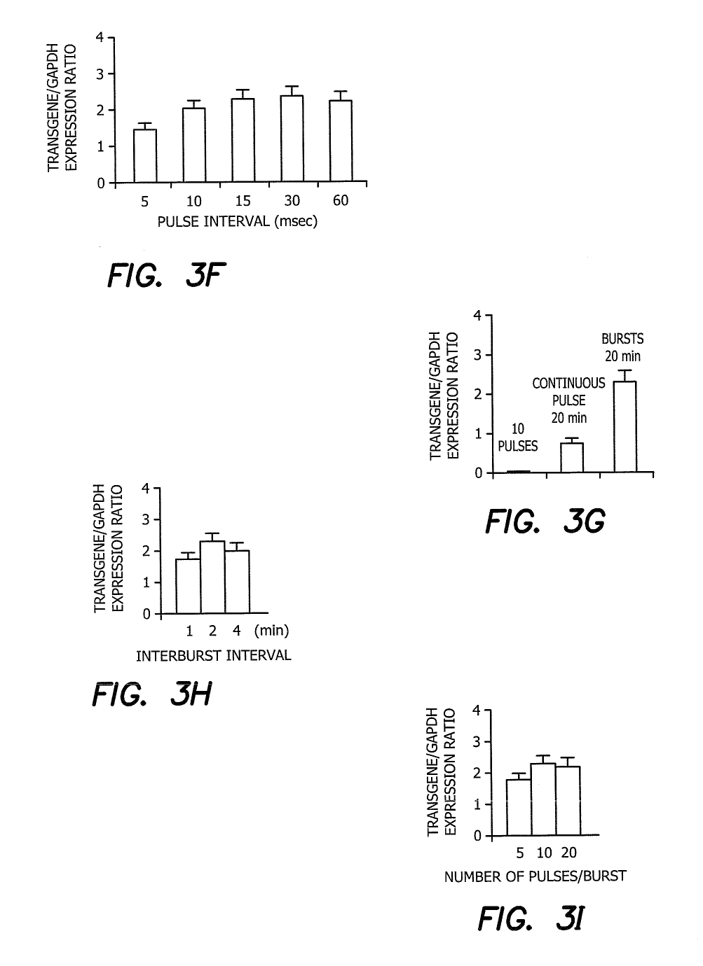

Although the optimized electric field in the present setting was 10 V/cm, only 25% decrease in the gene expression level was observed when 5 V/cm LSEN was applied as shown in the bar chart of FIG. 3a. This difference was gradually diminished in the long-term. At POD-8, the transgene expression level induced by 5 V/cm and 10 V/cm LSEN was similar as shown in the bar chart of FIG. 3b. The distribution of the transgene remained the same when 5 V/cm or 10 V/cm LSEN was applied. Further increase the electric field strength to 50 V/cm or above failed to improve the gene transfer efficiency. In contrast, it was diminished as seen in the comparative results in the charts of FIGS. 3a and 3b. In 100 V/cm treated hearts, necrotic cardiac myocytes and infiltrative cells were often observed in the electrodes contacted areas as shown in the comparative histological microphotographs of FIG. 3d.

The optimal pulse duration was 5-10 ms, which was shorter than in rat liver and mice skeletal muscle in vivo gene transfer as shown in FIG. 3e. This could be due to the better electrode-tissue conductance of LSEN system, or because of the higher electrical sensitivity of the myocardium. The optimal pulse interval was 15-30 ms as demonstrated in FIG. 3f. Our system allowed fresh plasmid-gene to continuously be delivered to each cell in the whole heart 18 and to dynamically interact with the cell membrane under the effect of an relatively uniform electric field network for a long time. The duration of 20 minutes was the shortest time that is allowed for performing any ex vivo treatment on a donor organ in a clinical heart transplantation, but it can be extended to several hours. Thus, we developed and validated a burst pulse protocol that has a long resting period between the bursts of pulses to allow the cell membrane to fully recover from the permeabilized state. We found that the transgene expression level in the heart 18 treated with the bursts of pulses with an optimal interburst interval more than 2.8-fold higher than that treated with uninterrupted rectangular pulse stimuli, and 48 times higher than that treated with a 10 pulse stimuli as shown in FIG. 3g. The optimized interburst interval was 2 minutes and the optimized pulse number was between 10 to 20 per burst as shown in FIGS. 3h and 3i respectively.

In summary, the strength of the electrical field is the most important parameter among others in determination of the gene transfer efficiency and tissue damage, followed by the length of the pulse. The number of the pulse has much less effect, and the number of burst only has a slight effect. Our study demonstrated that the optimal voltage for LSEN-mediated ex vivo gene transfer in large animal heart is 100- to 1000-fold lower than previously reported in vivo or in vitro gene transfer studies. Even in a recent study about in vivo skeletal muscle IL-5 gene transfer in mice with the lowest optimal voltage that has ever been reported, voltage level was still 50 times higher than here. There may be several possible reasons for this: 1) a cluster of cells has a better electrical conductance than cells in suspension, because the distance between the electrodes and the cell membrane is shorter, therefore lower voltage is required; 2) tissue with intact cell-to-cell connections has a better electrical conductance, and tissue which has a gap junction, such myocardium and skeletal muscle, has even better electrical conductance; 3) tissue with intact cell-to-cell connections and a gap junction might improve the homogeneity of the electric field distribution, so that cell damage may be reduced, and the gene expression increased; 4) our electropermeabilization array has much higher density of the electrodes, and better electrode-tissue contact, and has better uniformed electric field distribution. Genes infused through coronary artery also induces a much more uniformed gene distribution. Thus, efficient gene transfection can be induced by a low voltage electropermeabilization.

Turn now to an evaluation the possible cardiac adverse effects that might induced by low strength electric field network (LSEN). Our preliminary data suggest that the low strength electric field network-mediated gene transfer method of the illustrated embodiment seems more promising for the gene delivery in large animal and human organs than adenovirus- and liposome-mediated gene transfer techniques. It might also become an applicable protein and drug delivery strategy for cardiovascular diseases and other organic diseases. The safety issue of the electropermeabilization is always a major concern, especially for ex vivo and in vivo gene transfer. Cardiac tissue may be particularly amenable to electrical permeabilization, by virtue of its property as an electrical syncytium. On the other hand, the structural features of many voltage-sensitive proteins in the cardiac myocyte membrane suggest a high susceptibility to electric field induced electroconformational damage. Recent studies in electrical shock also suggest that electroporation induced high permeability to the ions could directly or indirectly affect both the electrical and mechanical activities of the myocytes. Although we are using very low voltage, it is crucial to systematically evaluate any possible cardiac adverse effect. Our functional cardiac isograft transplant rabbit model is the only model suitable for both hemodynamic and electrophysiologic study.

We evaluated the effect of LSEN on cardiac electrophysiology and hemodynamics in cardiac isografts using our rabbit heterotopic functional cervical heart transplants. We also compared the arrhythmogenic effect of LSEN-, adenovirus-, or liposome-mediated IL-10 gene transfers. In this model, as expected the acute or chronic allograft rejection was not observed in the 100-day observation period.

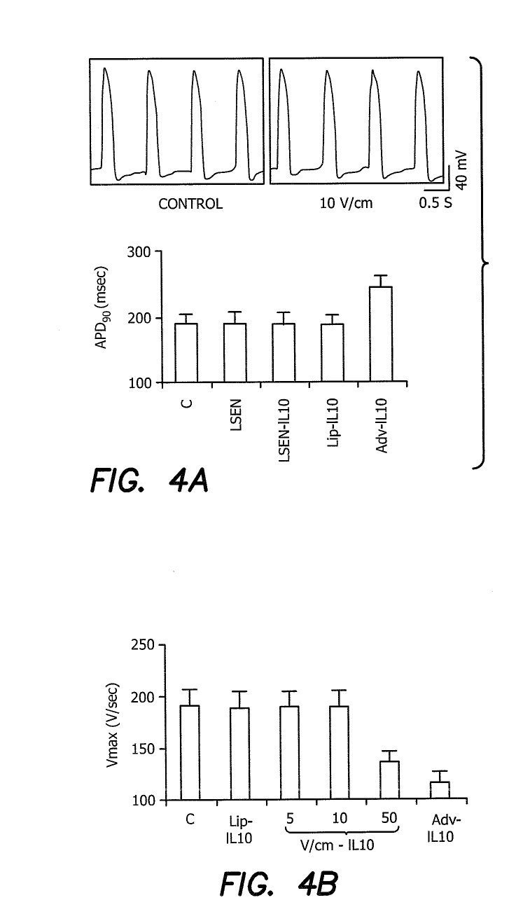

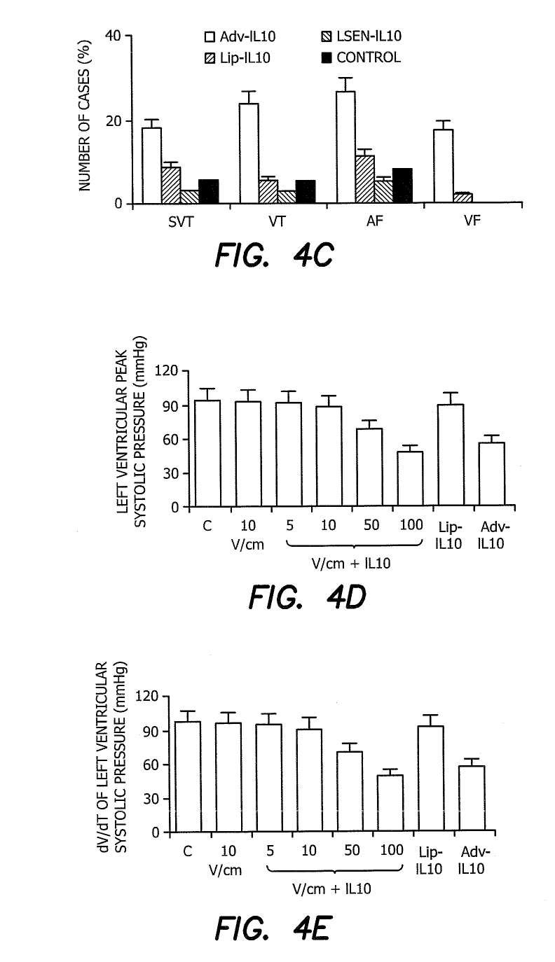

Consider now several different areas of examination and evaluation. The left ventricular endomyocardium monophasic action potential duration at 90% of repolarization (APD₉₀) was not changed in 5 or 10 V/cm LSEN or LSEN-phIL10 treated group compared with that in a sham operating group at 2 hours and 2 days after donor heart implantation as shown in FIG. 4a. An electrophysiologic recording was made continuously for 15 minutes. The amplitude and the maximum rate of rise of the action potential stroke (dV/dtₘₐₓ) also remained unchanged as shown in FIG. 4a and the bar chart of FIG. 4b. However, 50 V/cm LSEN significantly reduced the amplitude and d V/dtₘₐₓ of action potential and prolonged APD₉₀ in first 2-4 days and that was gradually recovered in following 2-4 weeks. Adenovirus-mediated IL-10 gene transfer caused significant prolongation of APD₉₀ at 2 hours, reduction of dV/dtₘₐₓ and greater APD₉₀ prolongation in long-term as shown in FIG. 4a and the bar chart of FIG. 4b, and also induced high incidence of atrial and ventricular arrhythmias as shown in FIG. 4c. Interestingly, the incidence of arrhythmias in LSEN and LSEN-phIL-10 gene treated groups was the same as that in sham group in first 2 hours. At POD 2, no arrhythmias observed in LSEN, LSEN-phIL-10 and liposome-phIL-10 groups during 2 hours recording. The peak and dV/dT of the left ventricular systolic pressure measured at POD 2 was not reduced in 5 or 10 V/cm LSEN-phIL-10 and liposome-phIL-10 treated groups as shown in FIGS. 4d and 4e. However, 50 V/cm or higher strength of LSEN causes significant decrease of the ventricular contractile function as shown in FIGS. 4d and 4e.

In summary, these results suggest that LSEN-mediated gene transfer has highest gene transfer efficiency, and has a most favorable safety profile compared with any virus or non-virus gene transfer approaches. These findings further confirmed the feasibility of this novel gene transfer approach.

Consider now the efficacy of low strength electric field network-mediated IL-10 gene therapy in a cardiac allograft rejection. We previously reported that adenovirus-mediated ex vivo gene transfer by intracoronary infusion resulted in an efficient vector uptake and intragraft over expression of immunosuppressive cytokine, IL-10, and doubled the longevity of allografts, but the transgene expression was transient, and had significant cardiac side effects, such as arrhythmogenic and negative inotropic effects. In contrast, liposome-mediated IL-10 transgene over expression was slowly initiated, but remained much longer and allograft survival was prolonged four fold. It has no cardiac side effects and did not generate the autoimmune response, but gene transfer efficiency was five times lower than adenovirus in the same model. The outcomes of both were still far from the completely satisfactory.

For a successful gene therapy, as previously stated there are two major requirements, one is an efficient gene transfer technique, and another of which is an effective candidate gene. LSEN-mediated ex vivo IL-10 gene transfer is not only highly efficient, but transgene expression is initiated early and long lasting without autoimmunogenecity and toxicity. We hypothesis that with the gene transfer strategy of the illustrated embodiment the efficacy of localized immunotherapy will be greatly improved and will be superior to that of any viral or other nonviral gene therapy.

Using the same rabbit heterotopic functional heart transplant model as described above, we again evaluated the therapeutic efficacy of low strength electropermeabilization-mediated IL-10 gene therapy in acute cardiac allograft rejection, and compared it with that in an adenovirus or liposome-mediated IL-10 gene therapy. Gene transfer was performed in the same way as that for isografts described above.

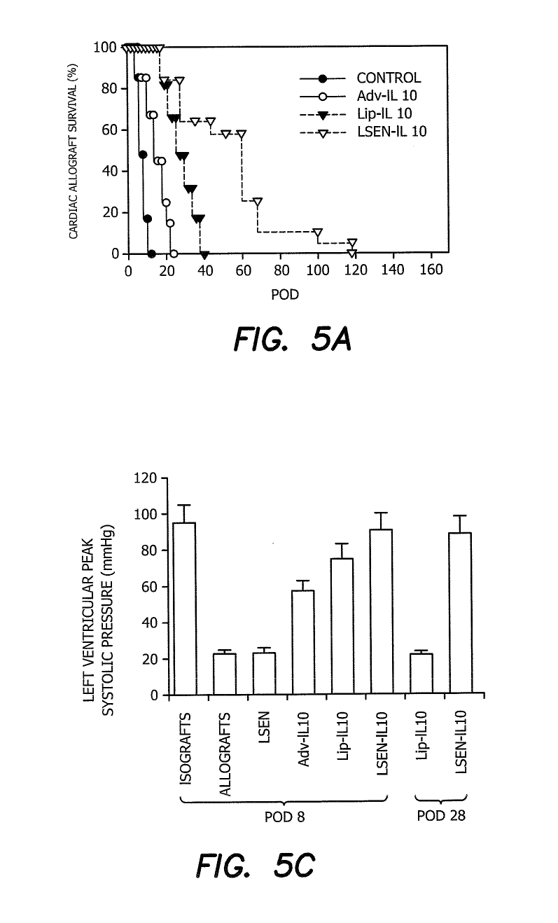

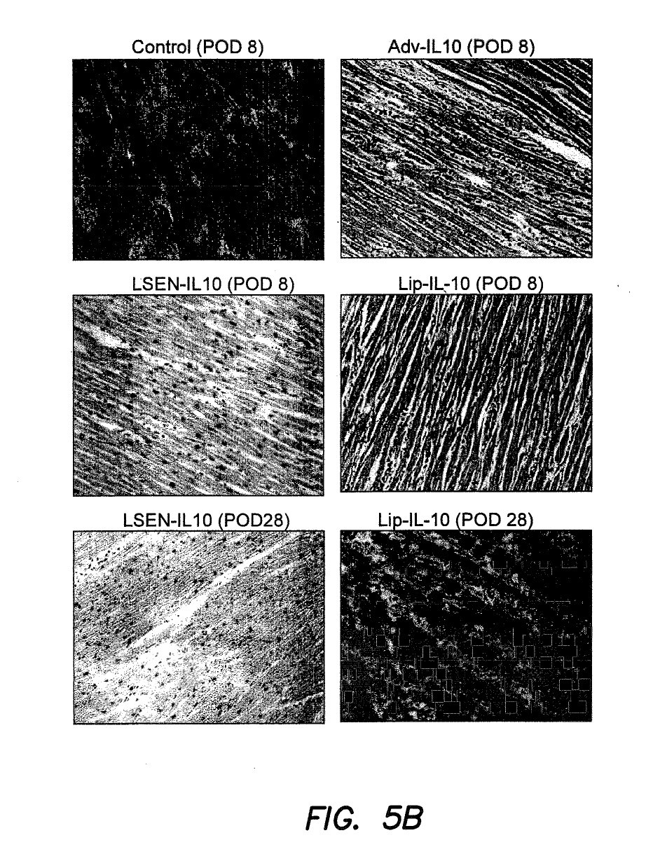

The gene transfer efficiency and the time course of transgene and protein expression in the cardiac allografts were the same as that seen in isografts described above (data not shown). LSEN-mediated ex vivo IL-10 gene transfer induced localized over expression of IL-10 that resulted in a significantly earlier and greater immunosuppression in the cardiac allografts compared with adenovirus- and liposome-mediated gene therapy. Allograft survival was further prolonged from 7±1 days in a sham group to and a LSEN-only treated group to 52±9 days in LSEN-IL-10 treated group. This is more than two fold longer than in liposome-mediated IL-10 gene therapy and more than four fold longer than that in adenovirus-mediated IL-10 gene therapy shown in FIG. 5a.

The rejection score was also greatly improved in LSEN-IL-10 gene therapy group compared with that in adenovirus- and liposome-mediated IL-10 gene therapy groups shown in comparative histological microphotographs of FIG. 5b. Left ventricular systolic pressure measured 2 hours after cardiac isografts were reestablished was not reduced by liposome-IL-10 gene infusion and 5 or 10 V/cm LSEN-mediated IL-10 gene transfer as shown in FIG. 5c. However, 50 V/cm or higher strength LSEN caused significant decrease of left ventricular contractile function compared with that in the controls. Additionally, a greater improvement of ventricular systolic function of cardiac allografts was observed in LSEN-mediated IL-10 gene therapy group compared to liposome and adenovirus-mediated IL-10 gene therapy group. The rejection score in electropermeabilization-IL10 group was significantly lower (2.0±0.3, p<0.05) than that of control group (3.7±0.4) in POD 3-6, and 1.8±0.3 in POD>31. The total of graft infiltrating cells was reduced 43% in POD 3-6 and 48% in POD>31, and the percentage of CD3+ T cells was significantly decreased (p<0.01) in POD 7-10.

Our results indicate that LSEN-mediated ex vivo intracoronary IL-10 gene transfer is able to induce the most efficient and uniformly distributed over expression of IL-10 in cardiac isografts and allografts. This strategy generated an earlier and more potent localized immunosuppression in cardiac allografts than that was seen in adenovirus or liposome-mediated IL-10 gene therapy. However, even with such a highly efficient and non-toxic/autoimmunogenic gene transfer strategy, true tolerance was still not achieved. These results suggest that IL-10 alone may be not sufficient to induce T cell anergy in cardiac allografts.

Consider now the efficiency of low strength electric field network-mediated ex vivo IL-4 and IL-10 combined gene transfer in rabbit cardiac allografts. Our previous study has shown liposome-mediated two plasmids, IL-4 and IL10, combined gene therapy in the same model. Localized over expression of IL-4 and IL-10 synergistically suppressed the alloimmune responses by significantly reducing T lymphocyte infiltration and cytoxicity, and promoted the long-term survival of cardiac allografts. We hypothesized that using LSEN we can induce a highly efficient and more balanced IL-4 and IL-10 over expression in cardiac allografts and may further improve the efficacy.

Recently, we have made a major breakthrough by using low strength electric field network to transfer a plasmid contains two therapeutic genes, IL-4 and IL-10 in rabbit heart 18. During gene infusion, the optimized low strength (LSEN) (10V/cm) electric field network was applied on the donor heart 18 as described above. Human recombinant IL-4 and IL-10 cDNAs driven by two identical CMV promoters in one vector was delivered ex vivo intracoronary into rabbit allografts.

LSEN is able induce a localized and balanced dual gene transfer in the targeted organ in contrast with the cationic liposome-mediated IL-4 and IL-10 combined gene transfer previously reported. The gene transfer efficiency was five times higher than liposome-mediated IL-10 gene transfer.

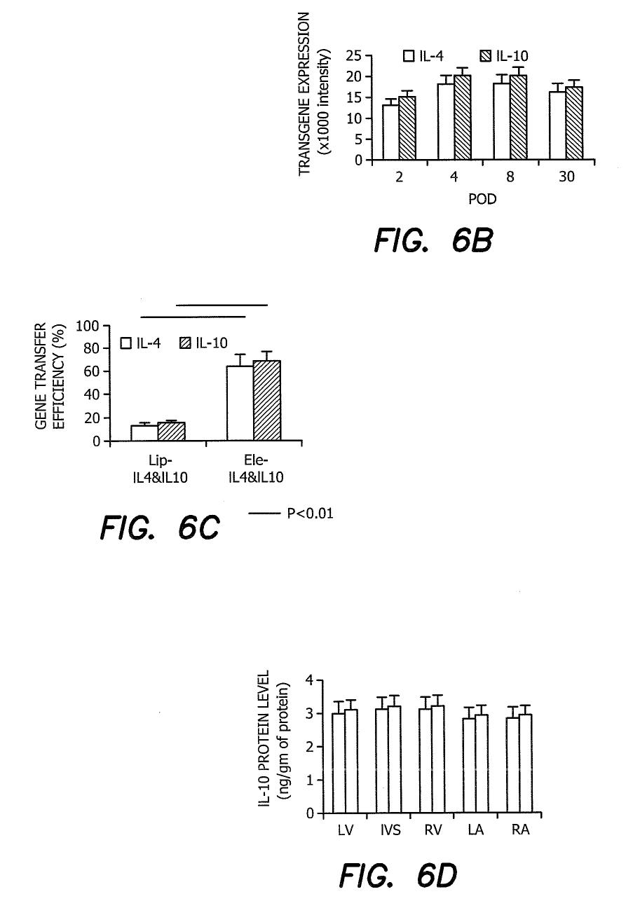

Both transfected genes were only expressed in the cardiac allografts, not in recipient's heart, brain, lung, liver, spleen, kidney and skeletal muscle. The amount and the time-course of IL-10 expression in the cardiac allografts remained the same in hIL-4 and hIL-10 combinatorial gene-transfer as that in the hIL-10 only gene transfer as shown in FIGS. 6a and 6b. The time course of hIL-4 transgene expression in the allografts was similar as hIL-10. The peak mRNA level of hIL-4 was slightly lower than hIL-10 in the cardiac allografts, but the difference between two genes was significantly smaller than that we previously reported in liposome-mediated IL-4 and IL-10 combined gene transfer. In that study IL-4 was driven by SV40 promoter, and IL-10 was driven by CMV promoter. The present results indicate that the significant low IL-4 gene expression occurred in our previous study are mainly due to the low output of SV40 promoter. The slightly low IL-4 gene expression in the present study might be due to the transcription nature of IL-4 itself, because this was also seen when we transfer IL-4 only, without IL-10. The time course of IL-10 mRNA expression in cardiac allografts was the same as that for IL-4. The efficiency of LSEN-mediated ex vivo hIL-4 and IL-10 combined gene transfer in cardiac allograft evaluated by in situ β-glactosidase staining was five times higher than that mediated by liposome as shown in the bar chart of FIG. 6c. The gene transfer efficiency for IL-4 was the same as IL-10, and was same as when they transferred alone. Most importantly, a balance IL4/IL10 protein expression was observed in cardiac allografts as shown in the bar chart of FIG. 6d. The IL-4 and IL-10 protein expression in LSEN-mediated combinatorial gene therapy was the same as that in IL-4 or 10 only gene transfer. Two genes transferred in a vector did not interfere each. Unlike mRNA expression, however, the decline of IL-4 and IL-10 protein expression was slower in combined gene therapy group than that in single gene therapy groups. The same phenomena was also observed in liposome-mediated IL-4 and IL10 combined gene therapy. The distribution of IL-4 and IL-10 was similar in all regions of the heart 18 as shown in FIG. 6d. There was no significant increase in IL-4 and IL-10 concentration in the recipients' serum, brain, lung, spleen, liver, kidney, and skeletal muscle in all time phases examined by ELISA, compared with those recipient rabbits treated with “empty” liposome (data not shown).

These results demonstrate that LSEN-mediated ex vivo gene transfer induces a balanced dual therapeutic transgene over expression in targeted organ while two genes are driven by two identical promoters and constructed in one vector.

Consider next the efficacy of low strength electric field network-mediated IL-4 and IL-10 combined gene therapy in cardiac allograft rejection. We hypothesize that with localized highly efficient and balanced IL-4 and IL-10 gene transfer, we should be able to further improve the efficacy of gene therapy for cardiac allograft rejection.

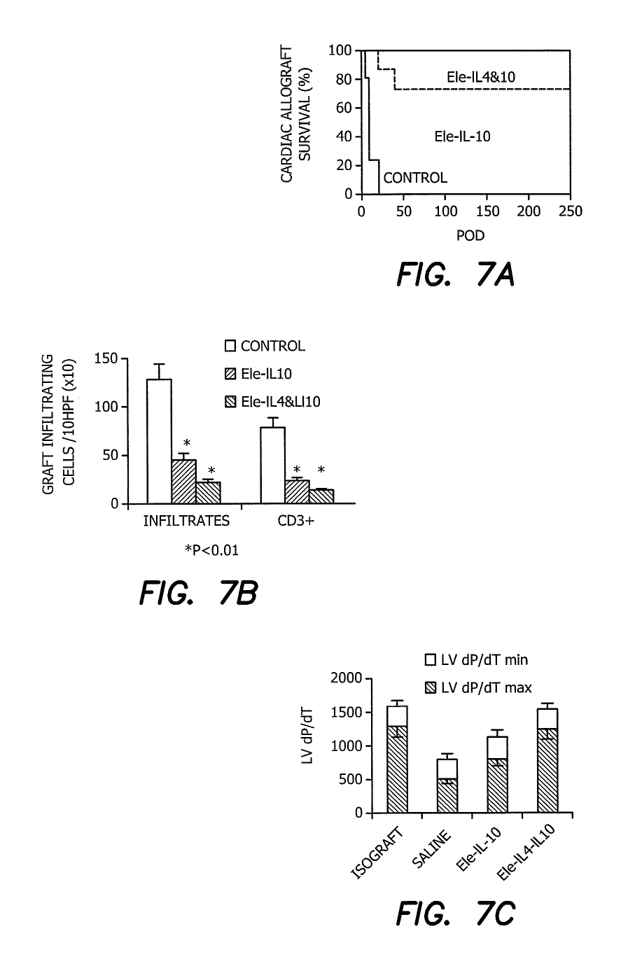

We examined the gene therapy effects on the allograft survival, function and immune responses. As shown in FIG. 7a, two thirds of the allografts achieved indefinite survivals. However, one third of the allografts failed around 2-3 weeks after operation. Half of them failed due to excessive effusion around the allograft and seroma formation. This never occurred in the electropermeabilization-mediated or liposome-mediated IL-10 gene transfer.

In the liposome-mediated IL-4 gene transfer, allografts only survived for 8±1 days due to sever acute rejection before seroma occurs (usually 2-3 weeks after operation). In liposome-mediated IL-4 and IL-10 combined gene transfer IL-4 expression level was 50% lower than IL-10, and sarcoma rarely occurred. In LSEN-mediated IL-4 and IL-10 combined gene transfer, the IL-4 protein level was only slightly lower than IL-10, but seroma occurred in 17% of the allografts. Over expressed IL-4 and IL-10 not only induced significant immunosuppression and T cell apoptosis, and also modulated the cytokine profile, and protected myocytes from apoptosis. The reduction of total amount of infiltrates and CD3+ T cells were significantly greater in LSEN-IL4 and IL10 gene therapy group compared with that in LSEN-IL10 treated allografts as shown in FIG. 7b. The percentage of TUNEL positive CD3+ T cells among total graft infiltrating CD3+ T cells on POD 7-8 was significantly (p<0.01) increased in the LSEN-mediated IL4 and IL10 gene therapy group (63% and 67%), respectively, compared with that in control group treated with antisense IL4 and IL10 genes (7% and 12%, respectively). The remarkable elevation of apoptotic T cells expressing Fas, FasL, Caspase-8 and Caspase-3 was revealed consistently in the IL-4 and IL-10 combined gene therapy group (data not shown). The LSEN-mediated IL4 and IL10 gene transfer significantly improved the cardiac function as shown in FIG. 7c. No arrhythmogenic effects and any other cardiac adverse effects were found.

These results demonstrate that early initiated and balanced excessive exogenous IL-4 and IL-10 expression induced by LSEN-mediated localized gene transfer in cardiac allografts has better immunosuppressive effect than LSEN-mediated IL-10 gene therapy or liposome-mediated IL-4 and IL-10 combined gene therapy. However, this approach is still not able to induce T cell anergy. Higher IL-4 expression promotes B cell activation may responsible for the seroma formation in the early stage.

Consider next the efficiency and efficacy of low strength electric field network-mediated IL-10 and CTLA4Ig combined gene therapy in cardiac allograft rejections. Previous studies have shown that CTLA4-Ig, a recombinant fusion protein that contains the extracellular domain of CTLA4 and Fc portion of IgG1, could strongly adhere to the B7 molecule to block CD28-mediated costimulatory signals. The engagement of antigen/major histocompatibility complex (MHC) with T cell receptor on helper T cells in absence of costimulatory signals induces T cell anergy, resulting in inhibition of in vitro and in vivo immune responses. In rodents, adenovirus-mediated CTLA4-Ig gene transfer prolongs allografts survival. Theoretically, CTLA4-Ig should have less B cell effect than IL-4, although it has never been systematically examined. It can be a candidate gene in combination with IL-10.

Using the same rabbit heart transplant model described above, we examined the efficiency and efficacy of LSEN-mediated human recombinant CTLA4-Ig and IL-10 combined gene therapy in acute cardiac rejection using the same protocol as we described above.

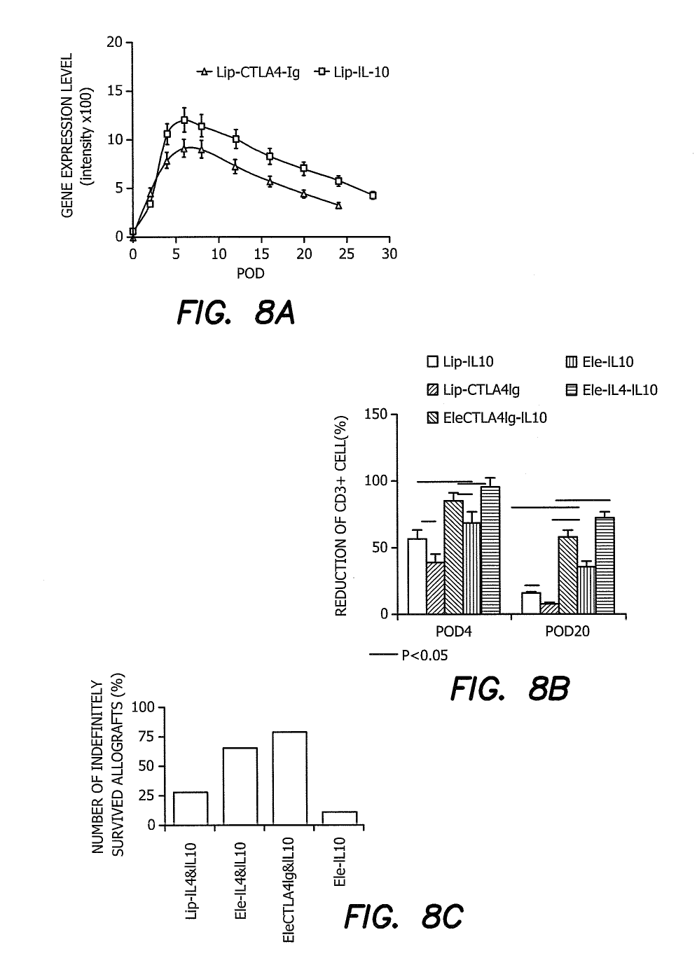

Previously, we compared the efficacy of ex vivo liposome-mediated human recombinant CTLA4-Ig to IL-10 gene therapy for acute cardiac rejection in the rabbit model. The time-course of CTLA4-Ig transgene expression was similar as IL-10. The gene transfer efficiency was slightly lower in CTLA4-Ig group than in IL-10 group as shown in FIG. 8a. CTLA4-Ig gene therapy significantly prolonged allograft survival from 9±2 days to 20±5 days. The allograft survival was shorter than IL-10 gene therapy, but longer than the IL-4 gene therapy.

While three doses of CTLA4-Ig gene were tested, 504, 1004 and 200 μg, the maximum therapeutic effect on the allograft rejection score and survival was induced by 100 μg. The reduction of the total number of infiltrating lymphocytes induced by CTLA4-Ig gene therapy was significantly less than that in IL-10 gene therapy group as shown in FIG. 8b, especially in the late stage. CTLA4-Ig gene therapy also promoted CD3+, CD4+ and CD8+ T cell apoptosis. The ratio of CD4+/CD8+ was slightly increased. Unlike IL-10, CTLA4-Ig gene therapy only slightly increased endogenous IL-4 and IL-10 gene expression (p<0.05), decreases IL-6 gene expression (p<0.05).

We also examined the efficiency of LSEN-mediated ex vivo CTLA-4 and IL-10 combined gene transfer in rabbit cardiac allografts. The peak expression level and time-course of IL-10 expression were similar as that in LSEN-IL-10 only gene transfer. CTLA4-Ig mRNA expression level was 17% lower than IL-10. Homogeneous distribution was observed as that in LSEN-mediated IL-10 gene transfer. As shown in FIG. 8b, over expression of both exogenous CTLA4-Ig and IL-10 induced by LSEN-mediated CTLA4-Ig and IL-10 combined gene transfer caused significant greater inhibitory effect on the CD3+ cells compared with IL-10 only gene transfer. At the later stage this synergistic effect was more pronounced. Reduction CD3+ cells was significantly less than that in LSEN-mediated or liposome-mediated IL4 and IL10 combined gene therapy (p<0.05). Although true T cell anergy was still not achieved, 78% of allografts survived indefinitely, which is significantly more than the LSEN-mediated IL-4 and IL-10 combined gene therapy (p<0.05, FIG. 8c). This outstanding outcome is because seroma only occurred in 1 out of 25 recipient rabbits. Allograft LV systolic pressure was slightly lower than that in electropermeabilization-mediated IL4 and IL10 gene therapy due to more lymphocyte infiltration. No arrhythmogenic or other cardiac adverse effect was observed.

These results demonstrate that LSEN is a most efficient and safe gene transfer method that has the potential for transferring two or more therapeutic genes into the large animal hearts. Combined immunosuppressive gene therapy is more effective than single gene therapy for allograft rejection. To induce T cell anergy and true tolerance, better gene combinations may be needed.

The illustrated embodiment is thus demonstrated to be an efficient and safe clinical applicable gene and siRNA targeting approach for the whole heart of large animal and human. This ex vivo low strength electric field network-mediated gene targeting strategy is also usable for protein and drug delivery in other organ, tissue and cell transplantation. The illustrated embodiment of the invention also can be used to develop new drugs for the prevention and treatment of allograft and xenograft rejection.

Organ transplantation is thought to be a curative therapy for various organ diseases. However, the allograft rejection remains a major obstacle for reaching its ultimate goal. Conventional systemic immunosuppression usually results in multiple, deleterious side effects requiring major dosage adjustments, and true tolerance is rarely achieved. More specific interventions at the level of lymphocyte priming and activation in the region of antigen presentation are expected for better outcome. The combinatorial drug and gene-based therapy disclosed above opens a new era for tolerance induction.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

I claim:

1. An improvement in a method for using a combination of a highly efficient low strength electric field network (LSEN) and an immunosuppressive drug, gene, siRNA and shRNA or other gene-based therapy to mediate at least one immune response within a donor organ, tissue or cells to prevent the acute and chronic rejection or to induce tolerance in a recipient whole body system comprising:
locally transferring at least one gene or a gene-based molecule in a plasmid ex vivo in a time interval between harvest and implantation of an allograft or xenograft before implantation to introduce the long-term over expression of at least one immunosuppressive and/or modulative molecule, or to down regulate at least one alloreactive molecule in the donor organ, tissue or cells only and not in the recipient's whole body system.

2. The improvement of claim 1 where locally transferring at least one gene or any other gene-based molecule in a plasmid ex vivo in the time interval between harvest and implantation of an allograft or xenograft before implantation comprises locally transferring at least two genes at the same time or at least two gene-based molecules in one plasmid or separate plasmids in any combination.

3. The improvement of claim 1 where locally transferring at least one gene or any other gene-based molecule in a plasmid ex vivo in the time interval between harvest and implantation of an allograft or xenograft before implantation comprises locally transferring more than two genes at the same time or more than two gene-based molecules in one plasmid or separate plasmids in any combination.

4. The improvement of claim 1 where locally transferring at least one gene or any other gene-based molecule in a plasmid ex vivo in the time interval between harvest and implantation of an allograft or xenograft before implantation comprises imposing an immune system mask on the donor organ, tissue or cells to greatly increase the therapeutic efficacy, and limit systemic side effects.

5. The improvement of claim 1 further comprising locally transferring at least one protein, antibody, drug and/or other molecule using LSEN in a plasmid ex vivo in the time interval between harvest and implantation of an allograft or xenograft before implantation.

6. The improvement of claim 1 where locally transferring at least one gene or a gene-based molecule in a plasmid ex vivo in the time interval between harvest and implantation of an allograft or xenograft before implantation comprises locally transferring one or more genes in one plasmid and locally transferring a drug or at least one other molecule simultaneously in another plasmid or by means not involving a plasmid.

7. A method comprising:
applying LSEN to a donor organ, tissue or cells; and
locally delivering before, during and/or after application of LSEN a drug, gene and siRNA or a gene-based therapeutic molecule or a combination thereof to the donor organ, tissue or cells to modulate immune responses within the donor organ, tissue or cells to prevent the acute and chronic rejection or induce tolerance in transplantation.

8. The method of claim 7 further comprising locally transferring at least one protein, antibody, drug and/or other molecule using LSEN in a plasmid ex vivo in the time interval between harvest and implantation of an allograft or xenograft before implantation.

9. The method of claim 7 where locally transferring at least one gene or a gene-based molecule in a plasmid ex vivo in the time interval between harvest and implantation of an allograft or xenograft before implantation comprises locally transferring one or more genes in one plasmid and locally transferring a drug or at least one other molecule simultaneously in another plasmid or by means not involving a plasmid.

10. An improvement in a method comprising:
applying low strength (≦10 v/cm) electric field network (LSEN) to a whole heart of large animal or human; and
transferring at least one gene into the cells of the whole heart of a large animal or human to induce a naked plasmid DNA transfer therein.

11. The method of claim 10 where using low strength (≦10 v/cm) electric field network (LSEN) to induce a naked plasmid DNA transfer comprises introducing at least one immunosuppressive molecule only into the graft, thereby limiting systemic side effects to prolong allograft survival, and simultaneously transferring at least one candidate gene for a particular disease.

12. The method of claim 10 further comprising locally transferring at least one protein, antibody, drug and/or other molecule using LSEN in a plasmid ex vivo in the time interval between harvest and implantation of an allograft or xenograft before implantation.

13. The method of claim 10 further comprising applying low strength (≦10 v/cm) electric field network (LSEN) to a heart for cardiac disease not involving transplantation and for any other organ transplantation for other organ diseases; and transferring at least one gene to the heart for the cardiac disease and for the other organ transplantation for other organ diseases to induce a plasmid DNA transfer therein.

14. The method of claim 10 where locally transferring at least one gene or a gene-based molecule in a plasmid ex vivo in the time interval between harvest and implantation of an allograft or xenograft before implantation comprises locally transferring one or more genes in one plasmid and locally transferring a drug or at least one other molecule simultaneously in another plasmid or by means not involving a plasmid.

15. The improvement of claim 1 further comprising locally transferring at least one molecule and combinations thereof for application in the transplantation of organs, tissues and cells selected from the group consisting of:
Cytokines:
Chemokines: CCL1, CCL11, CCL13, CCL16, CCL17, CCL18, CCL19, CCL2, CCL20, CCL21, CCL22, CCL23, CCL24, CCL25, CCL26, CCL27, CCL28, CCL3, CCL3L1, CCL4, CCL4L1, CCL5, CCL7, CCL8, CKLF, CX3CL1, CXCL1, CXCL10, CXCL11, CXCL12, CXCL13, CXCL14, CXCL2, CXCL3, CXCL5, CXCL6, CXCL9, CYP26B1, IL13, IL8, PF4V1, PPBP, PXMP2, XCL1;
Other Cytokines: AREG, BMP1, BMP2, BMP3, BMP7, CAST, CD40LG, CER1, CKLFSF1, CKLFSF2, CLC, CSF1, CSF2, CSF3, CTF1, CXCL16, EBI3, ECGF1, EDA, EPO, ERBB2, ERBB21P, FAM3B, FASLG, FGF10, FGF12, FIGF, FLT3LG, GDF2, GDF3, GDF5, GDF6, GDF8, GDF9, GLMN, GPI, GREM1, GREM2, GRN, IFNA1, IFNA14, IFNA2, IFNA4, IFNA8, IFNB1, IFNE1, IFNG, IFNK, IFNW1, IFNWP2, IK, IL10, IL11, IL12A, IL12B, IL15, IL16, IL17, IL17B, IL17C, IL17D, IL17E, IL17F, IL18, IL19, IL1A, IL1F10, IL1F5, IL1F6, IL1F7, IL1F8, IL1F9, IL1RN, IL2, IL20, IL21, IL22, IL23A, IL24, IL26, IL27, IL28B, IL29, IL3, IL32, IL4, IL5, IL6, IL7, IL9, INHA, INHBA, INHBB, KITLG, LASS1, LEFTY1, LEFTY2, LIF, LTA, LTB, MDK, MIF, MUC4, NODAL, OSM, PBEF1, PDGFA, PDGFB, PRL, PTN, SCGB1A1, SCGB3A1, SCYE1, SDCBP, SECTM1, SIVA, SLCO1A2, SLURP1, SOCS2, SPP1, SPRED1, SRGAP1, THPO, TNF, TNFRSF11B, TNFSF10, TNFSF11, TNFSF13, TNFSF13B, TNFSF14, TNFSF15, TNFSF18, TNFSF4, TNFSF7, TNFSF8, TNFSF9, TRAP1, VEGF, VEGFB, YARS;
Cytokine Receptors:
Cytokine Receptors: CNTFR, CSF2RA, CSF2RB, CSF3R, EBI3, EPOR, F3, GFRA1, GFRA2, GHR, IFNAR1, IFNAR2, IFNGR1, IFNGR2, IL10RA, IL10RB, IL11RA, IL12B, IL12RB1, IL12RB2, IL13RA1, IL13RA2, IL15RA, IL17R, IL17RB, IL18R1, IL1R1, IL1R2, IL1RAP, IL1RAPL2, IL1RL1, IL1RL2, IL20RA, IL21R, IL22RA1, IL22RA2, IL28RA, IL2RA, IL2RB, IL2RG, IL31RA, IL3RA, IL4R, IL5RA, IL6R, IL6ST, IL7R, IL8RA, IL8RB, IL9R, LEPR, LIFR, MPL, OSMR, PRLR, TTN;
Chemokine Receptors: BLR1, CCL13, CCR1, CCR10, CCR2, CCR3, CCR4, CCR5, CCR6, CCR7, CCR8, CCR9, CCRL1, CCRL2, CX3CR1, CXCR3, CXCR4, CXCR6, IL8RA, IL8RB, XCR1;
Cytokine Metabolism: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, IRF4, NALP12, PRG3, S100B, SFTPD, SIGIRR, SPN, TLR1, TLR3, TLR4, TLR6, TNFRSF7, TNFSF15;
Cytokine Production: APOA2, ASB1, AZU1, B7H3, CD28, CD4, CD80, CD86, EBI3, GLMN, IL10, IL12B, IL17F, IL18, IL21, IL27, IL4, INHA, INHBA, INHBB, INS, IRF4, NALP12, NFAM1, NOX5, PRG3, S100B, SAA2, SFTPD, SIGIRR, SPN, TLR1, TLR3, TLR4, TLR6, TNFRSF7;
Other Genes involved in Cytokine-Cytokine Receptor Interaction: ACVR1, ACVR1 B, ACVR2, ACVR2B, AMH, AMHR2, BMPR1A, BMPR1B, BMPR2, CCR1, CD40, CRLF2, CSF1R, CXCR3, IL18RAP, IL23R, LEP, TGFB1, TGFB2, TGFB3, TGFBR1, TGFBR2, TNFRSF1A, TNFRSF1B, TNFRSF21, TNFRSF8, TNFRSF9, XCR1;
Acute-Phase Response: AHSG, APCS, APOL2, CEBPB, CRP, F2, F8, FN1, IL22, IL6, INS, ITIH4, LBP, PAP, REG-III, SAA2, SAA3P, SAA4, SERPINA1, SERPINA3, SERPINF2, SIGIRR, STAT3;
Inflammatory Response: ADORA1, AHSG, AIF1, ALOX5, ANXA1, APOA2, APOL3, ATRN, AZU1, BCL6, BDKRB1, BLNK, C3, C3AR1, C4A, CCL1, CCL11, CCL13, CCL16, CCL17, CCL18, CCL19, CCL2, CCL20, CCL21, CCL22, CCL23, CCL24, CCL25, CCL26, CCL3, CCL3L1, CCL4, CCL4L1, CCL5, CCL7, CCL8, CCR1, CCR2, CCR3, CCR4, CCR7, CD14, CD40, CD40LG, CD74, CD97, CEBPB, CHST1, CIAS1, CKLF, CRP, CX3CL1, CXCL1, CXCL10, CXCL11, CXCL12, CXCL13, CXCL14, CXCL16, CXCL2, CXCL3, CXCL5, CXCL6, CXCL9, CYBB, DOCK2, EPHX2, F11 R, FOS, FPR1, GPR68, HDAC4, HDAC5, HDAC7A, HDAC9, HRH1, ICEBERG, IFNA2, IL10, IL10RB, IL13, IL17, IL17B, IL17C, IL17D, IL17E, IL17F, IL18RAP, IL1A, IL1B, IL1F10, IL1F5, IL1F6, IL1R1, IL1RAP, IL1RN, IL20, IL22, IL31 RA, IL5, IL8, IL8RA, IL8RB, IL9, IRAK2, IRF7, ITCH, ITGAL, ITGB2, KNG1, LTA4H, LTB4R, LY64, LY75, LY86, LY96, MEFV, MGLL, MIF, MMP25, MYD88, NALP12, NCR3, NFAM1, NFATC3, NFATC4, NFE2L1, NFKB1, NFRKB, NFX1, NMI, NOS2A, NR3C1, OLR1, PAP, PARP4, PLA2G2D, PLA2G7, PRDX5, PREX1, PRG2, PRG3, PROCR, PROK2, PTAFR, PTGS2, PTPRA, PTX3, REG-111, RIPK2, S100A12, S100A8, SAA2, SCUBE1, SCYE1, SELE, SERPINA3, SFTPD, SN, SPACA3, SPP1, STAB1, SYK, TACR1, TIRAP, TLR1, TLR10, TLR2, TLR3, TLR4, TLR5, TLR6, TLR7, TLR8, TLR9, TNF, TNFAIP6, TOLLIP, TPST1, VPS45A, XCR1;
Humoral Immune Response: BATF, BCL2, BF, BLNK, C1 R, C2, C3, C4A, CCL16, CCL18, CCL2, CCL20, CCL22, CCL3, CCL7, CCR2, CCR6, CCR7, CCRL2, CCRL2, CD1B, CD1C, CD22, CD28, CD40, CD53, CD58, CD74, CD86, CLC, CR1, CRLF1, CSF1R, CSF2RB, CXCR3, CYBB, EBI3, FADD, GP1, IL10, IL12A, IL12B, IL12RB1, IL13, IL18, IL1B, IL2, IL26, IL4, IL6, IL7, IL7R, IRF4, ITGB2, LTF, LY86, LY9, LY96, MAPK11, MAPK14, MCP, NFKB1, NR4A2, PAX5, POU2AF1, POU2F2, PTAFR, RFXANK, S100B, SERPING1, SFTPD, SLA2, TNFRSF7, XCL1, XCR1, YY1;
IL-1R/TLR Members and Related Genes:
Detection of Pathogens: TLR1, TLR3, TLR4, TLR6, TLR8.
Interleukin-1 Receptors: IL1R1, IL1R2, IL1RAP, IL1 RAPL2, IL1RL2.
Other Genes Involved in the IL-1R Pathway: IKBKB, MAPK14, MAPK8.
Inflammatory Response: IL1A, IL1B, IL1F10, IL1F5, IL1F6, IL1F8, IL1R1, IL1RN, IRAK2, MYD88, NFKB1, TLR1, TLR10, TLR2, TLR3, TLR4, TLR6, TLR8, TLR9, TNF, TOLLIP;
Apoptosis: IL1A, IL1B, NFKB1, NFKBIA, TGFB1, TNF;
Cytokines: IFNA1, IFNB1, IL1A, IL1B, IL1F10, IL1F5, IL1F6, IL1F7, IL1F8, IL1F9, IL6, TNF;
Genes Involved in NFκB Signaling: CHUK, IRAK2, MYD88, TLR1, TLR3, TLR4, TLR6, TLR8, TRAF6;
Host Defense to Bacteria:
Detection of Bacteria: CD1 D, PGLYRP1, PGLYRP2, PGLYRP3, TLR1, TLR3, TLR6;
LSP Receptor: CD14, CXCR4, DAF;
Acute-phase Response: CRP, FN1, LBP;
Complement Activation: C5, C8A, DAF, PFC;
Inflammatory Response: AZU1, C5, CCL2, CD14, CRP, CYBB, LY96, NFKB1, NOS2A, PRG2, S100A12, STAB1, TLR1, TLR3, TLR6, TLR9;
Cytokines, Chemokines, and their Receptors: C5, CCL2, CXCR4, IFNGR1, IFNGR2, IL12RB2, PPBP;
Antibacterial Humoral Response: CLECSF12, COLEC12, CYBB, DEFA5, DEFA6, LY96, NFKB1;
Defense Response to Bacteria: AZU1, BPI, CAMP, CLECSF12, DCD, DEFA4, DEFA5, DEFA6, DEFB1, DEFB118, DEFB127, DEFB4, GNLY, HAMP, LALBA, LBP, LEAP-2, LTF, LYZ, NOS2A, PFC, PGLYRP1, PGLYRP2, PGLYRP3, PPBP, PRG2, RNASE3, RNASE7, S100A12, STAB1, TLR3, TLR6, TLR9;
Other Genes Involved in the Host Defense Against Bacteria: CARD12, CHIT1, DMBT1, HAT, IRF1, NCF4, NFKBIA, PLUNC, SLC11A1;
Innate Immune Response:
Innate Immune Response: APOBEC3G, COLEC12, CRISP3, DEFB1, DEFB118, DEFB127, DMBT1, PGLYRP1, PGLYRP2, PGLYRP3, PLUNC, RNASE7, SFTPD, TLR8;
Other Genes Involved in the Innate Immune Response: ARTS-1, CD1D, IFNB1, IFNK, KIR3DL1, TLR10;
Septic Shock:
Apoptosis: ADORA2A, CASP1, CASP4, IL10, IL1B, NFKB1, PROC, TNF, TNFRSF1A;
Cytokines and Growth Factors: CSF3, IL10, IL1B, IL6, MIF, TNF;
Inflammatory Response: ADORA2A, CCR3, IL10, IL1B, IL1RN, MIF, NFKB1, PTAFR, TLR2, TLR4, TNF;
Other Genes Involved in Septic Shock: GPR44, HMOX1, IRAK1, NFKB2, SERPINA1, SERPINE1, TREM1;
B-cell activation:
Antigen dependent B-cell activation: CD28, CD4, CD80, HLA-DRA, IL10, IL2, IL4, TNFRSF5, TNFRSF6, TNFSF5, TNFSF6;
Other genes involved in B-cell activation: BLR1, HDAC4, HDAC5, HDAC7A, HDAC9, ICOSL, IGBP1, MS4A1, RGS1, SLA2;
B-cell proliferation: CD81, IFNB1, MO, TNFRSF5, TNFRSF7, TNFSF5;
B-cell differentiation: A1CDA, BLNK, GALNAC4S-6ST, HDAC4, HDAC5, HDAC7A, HDAC9, IL10, IL11, IL4, INHA, INHBA, KLF6, TNFRSF7;
B-cell activation:
Regulators of T-cell activation: CD2, CD3D, CD3E, CD3G, CD4, CD7, CD80, CD86, CD8A, CD8B1, CLECSF12, ICOSL, IRF4, KIF13B, NCK1, NCK2, PRLR, SIT, SLA2, TNFSF14;
T-cell proliferation: CD28, CD3E, GLMN, ICOSL, IL10, IL12B, IL18, IL27, NCK1, NCK2, SFTPD, SPP1, TNFSF14;
T-cell differentiation: CD1D, CD2, CD4, CD80, CD86, IL12B, IL2, IL27, IRF4, JAG2, NOS2A, RHOH, SOCS5, TNFRSF7, WWP1;
Regulators of Th1 and Th2 development: ANPEP, CD2, CD33, CD5, CD7, CSF2, IFNA2, IFNB1, IFNG, IL10, IL12A, IL13, IL3, IL4, IL5, ITGAX, TLR2, TLR4, TLR7, TLR9, TNFRSF5;
Genes involved in Th1/Th2 differentiation: CD28, CD86, HLA-DRA, IFNG, IFNGR1, IFNGR2, IL12A, IL12B, IL12RB1, IL12RB2, IL18, IL18R1, IL2, IL2RA, IL4, IL4R, PVRL1, TNFRSF5, TNFSF5;
Genes involved in T-cell polarization: CCL3, CCL4, CCR1, CCR2, CCR3, CCR4, CCR5, CCR7, CD28, CD4, CSF2, CXCR3, CXCR4, IFNG, IFNGR1, IFNGR2, IL12A, IL12RB1, IL12RB2, IL18R1, IL2, IL4, IL4R, IL5, TGFB1, TNFSF5;
Other genes related to immune cell activation:
Macrophage activation: C1QR1, IL31RA, INHA, INHBA, TLR1, TLR4, TLR6;
Neutrophil activation: APOA2, IL8, PREX1, PRG3;
Natural killer cell activation: CD2, IFNB1, IFNK, IL12B, IL2, IL21R, KIR3DL1, ULBP1, ULBP2, ULBP3;
Others: AZU1, CX3CL1, ITIH1, TOLLIP, TXNDC, ZNF3;
B-cell activation:
Antigen dependent B-cell activation: CD28, CD4, CD80, HLA-DRA, IL10, IL2, IL4, TNFRSF5, TNFRSF6, TNFSF5, TNFSF6;
Other genes involved in B-cell activation: BLR1, HDAC4, HDAC5, HDAC7A, HDAC9, ICOSL, IGBP1, MS4A1, RGS1, SLA2;
B-cell proliferation: CD81, IFNB1, IL10, TNFRSF5, TNFRSF7, TNFSF5;
B-cell differentiation: AICDA, BLNK, GALNAC4S-6ST, HDAC4, HDAC5, HDAC7A, HDAC9, IL10, IL11, IL4, INHA, INHBA, KLF6, TNFRSF7;
T-cell activation:
Regulators of T-cell activation: CD2, CD3D, CD3E, CD3G, CD4, CD7, CD80, CD86, CD8A, CD8B1, CLECSF12, ICOSL, IRF4, KIF13B, NCK1, NCK2, PRLR, SIT, SLA2, TNFSF14;
T-cell proliferation: CD28, CD3E, GLMN, ICOSL, IL10, IL12B, IL18, IL27, NCK1, NCK2, SFTPD, SPP1, TNFSF14;
T-cell differentiation: CD1D, CD2, CD4, CD80, CD86, IL12B, IL2, IL27, IRF4, JAG2, NOS2A, RHOH, SOCS5, TNFRSF7, WWP1;
Regulators of Th1 and Th2 development: ANPEP, CD2, CD33, CD5, CD7, CSF2, IFNA2, IFNB1, IFNG, IL10, IL12A, IL13, IL3, IL4, IL5, ITGAX, TLR2, TLR4, TLR7, TLR9, TNFRSF5;
Genes involved in Th1/Th2 differentiation: CD28, CD86, HLA-DRA, IFNG, IFNGR1, IFNGR2, IL12A, IL12B, IL12RB1, IL12RB2, IL18, IL18R1, IL2, IL2RA, IL4, IL4R, PVRL1, TNFRSF5, TNFSF5;
Genes involved in T-cell polarization: CCL3, CCL4, CCR1, CCR2, CCR3, CCR4, CCR5, CCR7, CD28, CD4, CSF2, CXCR3, CXCR4, IFNG, IFNGR1, IFNGR2, IL12A, IL12RB1, IL12RB2, IL18R1, IL2, IL4, IL4R, IL5, TGFB1, TNFSF5;
Other genes related to immune cell activation:
Macrophage activation: C1QR1, IL31RA, INHA, INHBA, TLR1, TLR4, TLR6.
Neutrophil activation: APOA2, IL8, PREX1, PRG3;
Natural killer cell activation: CD2, IFNB1, IFNK, IL12B, IL2, IL21R, KIR3DL1, ULBP1, ULBP2, ULBP3;
Others: AZU1, CX3CL1, ITIH1, TOLLIP, TXNDC, ZNF3;
CTGFβ Superfamily Cytokines:
TGF-β: TGFB1, TGFB2, TGFB3;
BMP: BMP1, BMP2, BMP3, BMP4, BMP5, BMP6, BMP7, BMP8B, BMP10, BMP15;
GDF: AMH, GDF1, GDF2 (BMP9), GDF3 (Vgr-2), GDF5 (CDMP-1), GDF6, GDF7, GDF8, GDF9, GDF10, GDF11 (BMP11), GDF15, IGF1, IGFBP3, IL6, INHA (inhibin a), INHBA (inhibin BA), IVL (involucrin), LEFTY1, LEFTY2, LTBP1, LTBP2, LTBP4, NODAL, PDGFB, TDGF1;
Activin: INHA (inhibin a), INHBA (inhibin BA), INHBB (inhibin BB), INHBC (inhibin BC), INHBE, LEFTY1, LEFTY2, NODAL;
Receptors: ACVR1 (ALK2), ACVR1 B (ALK4), ACVR1C, ACVR2, ACVR2B, ACVRL1 (ALK1), AMHR2, BMPR1A (ALK3), BMPR1B (ALK6), BMPR2, ITGB5 (integrin B5), ITGB7 (integrin B7), LTBP1, MAP3K7IP1, NROB1, STAT1, TGFB1I1, TGFBR1 (ALK5), TGFBR2, TGFBR3, TGFBRAP1;
SMAD: SMAD1 (MADH1), SMAD2 (MADH2), SMAD3 (MADH3), SMAD4 (MADH4), SMAD5 (MADH5), SMAD6 (MADH6), SMAD7 (MADH7), SMAD9 (MADH9);
SMAD Target Genes:
TGF-βActivin-responsive: CDC25A, CDKN1A (p21WAF1/p21CIP1), CDKN2B (p15LNK2B), COL1A1, COL1A2, COL3A1, FOS, GSC (goosecoid), IGF1, IGFBP3, IL6, ITGB5 (integrin B5), ITGB7 (integrin B7), IVL (involucrin), JUN, JUNB, MYC, PDGFB, SERPINE 1 (PAI-1), TGFB1I1, TGFB1I4, TGFBI, TGIF, TIMP1;
BMP-Responsive: BGLAP (osteocalcin), DLX2, ID1, ID2, ID3, ID4, JUNB, SMAD6 (MADH6), SOX4, STAT1, TCF8;
Molecules Regulating Signaling of the TGF-β Superfamily: BAMBI, BMPER, CDKN2B (p15LNK2B), CER1 (cerberus), CHRD (chordin), CST3, ENG (Evi-1), EVI1, FKBP1B, FST (follistatin), GREM1, HIPK2, MAP3K7, NBL1 (DAN), NOG, PLAU (uPA), RUNX1 (AML1), RUNX2, SMURF1, SMURF2, TDGF1;
Adhesion and Extracellular Molecules:
< > BGLAP (osteocalcin), ENG (Evi-1), ITGB5 (integrin B5), ITGB7 (integrin B7), TGFB1I1, TGFB1;
Extracellular Matrix Structural Constituents: BGLAP (osteocalcin), COL1A1, COL1A2, COL3A1, IVL (involucrin), LTBP1, LTBP2, LTBP4, TGFB1, TIMP1;
Other Extracellular Molecules: AMH, BMP1, BMP10, BMP15, BMP2, FST (follistatin), GDF1, GDF10, GDF15, GDF2 (BMP9), GDF3 (Vgr-2), GDF9, GREM1, IGF1, IGFBP3, IL6, INHA (inhibin a), INHBA (inhibin BA), INHBB (inhibin BB), INHBC (inhibin BC), PDGFB, PLAU (uPA), SERPINE1;
Transcription Factors and Regulators: DLX2, EVI1, FOS, GSC (goosecoid), HIPK2, ID1, ID3, ID4, JUN, JUNB, MYC, NROB1, RUNX1 (AML1), RUNX2, SMAD1 (MADH1), SMAD2 (MADH2), SMAD3 (MADH3), SMAD4 (MADH4), SMAD5 (MADH5), SMAD6 (MADH6), SMAD7 (MADH7), SMAD9 (MADH9), SMURF2, SOX4, STAT1, TCF8 (AREB6), TGFB1I1, TGFB1I4, TGIF;
Genes Involved in Cellular and Developmental Processes:
Apoptosis: CDKN1A (p21WAF1/p21CIP1), HIPK2, IGFBP3, INHA (inhibin a), INHBA (inhibin BA), STAT1, TDGF1, TGFB1;
Embryonic Development: BMP10, BMP4, GDF11 (BMP11), INHBA (inhibin BA), SMAD3, SMURF1, TDGF1;
Muscle Development: GDF8, GDF9, IGF1, SMAD3;
Neurogenesis: DLX2, GDF11 (BMP11), GREM1, INHA (inhibin a), INHBA (inhibin BA), NOG;
Reproduction: AMH, AMHR2, BMP15, FST (follistatin), GDF9, INHA (inhibin a), INHBA (inhibin BA), INHBB (inhibin BB), INHBC (inhibin BC), LEFTY2, NROB1, TDGF1;
Skeletal Development: BGLAP (osteocalcin), BMP1, BMP2, BMP3, BMP4, BMP5, BMP6, BMP7, BMP8B, BMPR2, CHRD (chordin), COL1A1, COL1A2, GDF10, GDF11 (BMP11), IGF1, INHA (inhibin a), INHBA (inhibin BA), NOG, RUNX2;
TH1 Cytokines and Related Genes: CCR5, CD28, CSF2 (GM-CSF), CXCR3, HAVCR2 (TIM3), IFNG, IGSF6 (CD40L), IL12B, IL12RB2, IL18, IL18BP, IL18R1, IL2, IL2RA (CD25), IRF1, SOCS1 (SSI-1), SOCS5, STAT1, STAT4, TBX21 (T-bet), TNF;
TH2 Cytokines and Related Genes: CCL11 (eotaxin), CCL15 (MIP-1d), CCL5 (RANTES), CCL7 (MCP-3), CCR2 (MCP-1), CCR3, CCR4, CCR9, CEBPB, FLJ14639 (NIP45), GATA3, GFI1, GPR44 (CRTH2), ICOS, IL10, IL13, IL13RA1, IL13RA2, IL1R1, IL1R2, IL4, IL4R, IL5, IL9, IRF4, JAK1, JAK3, MAF, NFATC1 (NFATc), NFATC2 (NFATp), NFATC3 (NFAT4), NFATC4, RNF110 (ZNF144), STATE, TLR4, TLR6, TMED1, ZFPM2 (FOG2);
CD4+ T Cell Markers: BCL3 (p50), CD4, CD69, CD80, CD86, CREBBP (CBP), CTLA4, IL15, IL6, IL6R, IL7, JAK2, LAG3, LAT, MAP2K7 (JNKK2), MAPK10 (JNK-3), MAPK8 (JNK-1), MAPK9 (JNK-2), PTPRC (CD45), SOCS3 (SSI-3), TFCP2 (CP2), TGFB3, TNFRSF21 (DR6), TNFRSF7 (CD27), TNFRSF8 (CD30), TNFRSF9 (4-1 BB), TNFSF4 (OX-40), TNFSF5 (CD40), TNFSF6 (FasL), TYK2, YY1;
Immune Cell Activation:
T-cell Activation: CD2, CD28, CD4, CD80, CD86, GLMN, IL10, IL12B, IL18, IL2, IL27, IRF4, SFTPD, SOCS5, SPP1, TNFRSF7 (CD27);
B-cell Activation: IL10, IL4, INHA, INHBA, TNFRSF7 (CD27), TNFSF5 (CD40);
T-helper 1 Type Immune Response: CD4, CD80, CD86, GLMN, IL10, IL17F, IL18, IL18BP, INHA, INHBA, IRF4, SFTPD, SPP1, TLR4, TLR6, IL12B, IL27, TNFRSF7 (CD27);
T-helper 2 type Immune Response: CD86, IL10, IL18, IL4, IRF4;
Antimicrobial Humoral Response: CCL15 (MIP-1d), CCL7 (MCP-3), CCR2 (MCP-1), CXCR3, FADD (Fas), IL12B, IL13, NFKB1, SFTPD, YY1;
Other Immune Response Genes: CSF2 (GM-CSF), FOSL1 (Fra-1), CEBPB, FOS, IRF1, MHC2TA (CIITA), SOCS6;
Transcription Factors and Regulators:
Positive Regulation of Transcription: CD80, CD86, IRF4;
RNA Polymerase II Transcription Factor Activity: ATF2, FOS, GFI1, IRF4, JUN, JUNB, JUND, MAF, MHC2TA (CIITA);
Transcription Co-activator Activity: ATF2, CREBBP (CBP), JUNB, MHC2TA (CIITA), NFATC3, NFATC4, YY1;
Transcription Co-repressor Activity: JUNB, YY1;
Transcription Factor Activity: CEBPB, CREBBP (CBP), FOSL1 (Fra-1), FOSL2 (Fra-2), GATA3, IRF1, JUND, NFATC1(NFATc), NFATC2 (NFATp), NFATC3 (NFAT4), NFATC4, NFKB1, RNF110 (ZNF144), STAT1, STAT4, STATE, TBX21 (T-bet), TFCP2 (CP2), YY1;
Transcription from Pal II Promoter: CEBPB, FOSL1 (Fra-1), GATA3, IRF1, MAF, NFATC1, NFATC3, NFATC4, NFKB1, STAT1;
Other Transcription Factors and Requlators: BCL3 (p50), JUN (c-JUN), SOCS2 (STATI2), SOCS4 (CIS4), SOCS6, SOCS7 (SOCS4), TH1L, TNF, ZFPM2 (FOG2);
Toll-Like Receptors: LY64 (RP105/CD180), SIGIRR (TIR8), TLR1, TLR2, TLR3, TLR4, TLR5, TLR6, TLR7, TLR8, TLR9, TLR10;
Adaptors & TLR Interacting Proteins: BTK, CD14, GPC1 (SP-A), HMGB1, HRAS, HSPA1A, HSPA4, HSPA6, HSPD1, LY86 (MD-1), LY96, MAL, MAPK81P3 (JIP3), MYD88, PELI1 (Pellino 1), PELI2 (Pellino 2), PGLYRP1, PGLYRP2, PGLYRP3, PGLYRPIbeta, RIPK2 (RIP2), SARM1, TICAM2, TIRAP, TOLLIP, TRIF (TICAM1);
Effectors: CASP8, EIF2AK2, FADD, IRAK1, IRAK2, IRAK3, IRAK4, MAP3K7, MAP3K7IP1 (TAB1), MAP3K7IP2 (TAB2), NR2C2 (TAK1), PPARA, PRKRA (PKR), SITPEC (ECSIT), TRAF6, UBE2N (Ubc13), UBE2V1 (Uev1A);
Downstream Pathways and Target Genes:
NFκB Pathway: CCL2 (MCP-1), CHUK (IKK-a), CSF2 (GM-CSF), CSF3 (G-CSF), IFNB1, IFNG, IKBKB (IKK-b), IKBKG (IKK-g), IL1A, IL1B, IL2, IL6, IL8, IL10, IL12A, IL12B, LTA (TNF-b), MAP3K1 (MEKK1), MAP3K14, MAP4K4 (NIK), NFKB1, NFKB2, NFKBIA (IkBa/mad3), NFKBIB (IkBb), NFKBIE, NFKBIL1, NFKBIL2, NFRKB, REL, RELA, RELB, TNF (TNFa), TNFRSF1A, TRADD;
JNK/p38 Pathway: ELK1, FOS, JUN, MAP2K3 (MKK3), MAP2K4 (MKK4), MAP2K6 (MKK6), MAP3K1 (MEKK1), MAPK8 (JNK1), MAPK9 (JNK2), MAPK10, MAPK11 (p38bMAPK), MAPK12 (p38gMAPK), MAPK13, MAPK14 (p38 MAPK);
NF/IL6 Pathway: CLECSF9, PTGES, PTGS2 (Cox-2);
IRE Pathway: CXCL10 (IP-10), IFNB1, IFNG, IRF1, IRF3, IRF7, TBK1;
Regulation of Adaptive Immunity: CD80, CD86, RIPK2 (RIP2), TRAF6;
Growth factor and associated molecule: BMP1, BMP2, BMP3, BMP4, BMP5, BMP6, BMP7, BMP8, BMPR1A, CASR, CSF2 (GM-CSF), CSF3 (G-CSF), EGF, EGFR, FGF1, FGF2, FGF3, FGFR1, FGFR2, FGFR3, FLT1, GDF10, IGF1, IGF1R, IGF2, MADH1, MADH2, MADH3, MADH4, MADH5, MADH6, MADH7, MADH9, MSX1, MSX2, NFKB1, PDGFA, RUNX2 (CBFA1), SOX9, TGFB1, TGFB2, TGFB3, TGFBR1, TGFBR2, TNF (TNFa), TWIST, VDR, VEGF, VEGFB, VEGFC;
Matrix and its associated protein: ALPL, ANXA5, ARSE, BGLAP (osteocalcin), BGN, CD36, CD36L1, CD36L2, COL1A1, COL2A1, COL3A1, COL4A3, COL4A4, COL4A5, COL5A1, COL7A1, COL9A2, COL10A1, COL11A1, COL12A1, COL14A1, COL15A1, COL16A1, COL17A1, COL18A1, COL19A1, CTSK, DCN, FN1, MMP2, MMP8, MMP9, MMP10, MMP13, SERPINH1 (CBP1), SERPINH2 (CBP2), SPARC, SPP1 (osteopontin);
Cell adhesion molecule: ICAM1, ITGA1, ITGA2, ITGA3, ITGAM, ITGAV, ITGB1, VCAM1;
Cell Growth and Differentiation:
Regulation of the Cell Cycle: EGFR, FGF1, FGF2, FGF3, IGF1 R, IGF2, PDGFA, TGFB1, TGFB2, TGFB3, VEGF, VEGFB, VEGFC;
Cell Proliferation: COL18A1, COL4A3, CSF3, EGF, EGFR, FGF1, FGF2, FGF3, FLT1, IGF1, IGF1R, IGF2, PDGFA, SMAD3, SPP1, TGFB1, TGFB2, TGFB3, TGFBR2, VEGF, VEGFB, VEGFC;
Growth Factors and Receptors: BMP1, BMP2, BMP3, BMP4, BMP5, BMP6, BMP7, BMP8B, BMPR1A, CSF2, CSF3, EGF, EGFR, FGF1, FGF2, FGF3, FGFR1, FGFR2, FGFR3, FLT1, GDF10, IGF1, IGF1 R, IGF2, PDGFA, SPP1, TGFB1, TGFB2, TGFB3, TGFBR1, TGFBR2, VEGF, VEGFB, VEGFC;
Cell Differentiation: SPP1, TFIP11, TWIST1, TWIST2;
Extracellular Matrix (ECM) Molecules:
Basement Membrane Constituents: COL4A3, COL4A4, COL4A5, COL7A1, SPARC;
Collagens: COL10A1, COL11A1, COL12A1, COL14A1, COL15A1, COL16A1, COL18A1, COL19A1, COL1A1, COL1A2, COL2A1, COL3A1, COL4A3, COL4A4, COL4A5, COL5A1, COL7A1, COL9A2;
ECM Protease Inhibitors: AHSG, COL4A3, COL7A1, SERPINH1;
ECM Proteases: BMP1, CTSK, MMP10, MMP13, MMP2, MMP8, MMP9, PHEX;
Structural Constituents of Bone: BGLAP, COL1A1, COL1A2, MGP;
Structural Constituents of Tooth Enamel: AMBN, AMELY, ENAM, STATH, TUFT1;
Other ECM Molecules: BGN, BMP2, BMP8B, COL17A1, COMP, CSF2, CSF3, DCN, DSPP, EGF, FGF1, FGF2, FGF3, FLT1, GDF10, IBSP, IGF1, IGF2, PDGFA, SPP1, VEGF, VEGFB;
Cell Adhesion Molecules:
Cell-cell Adhesion: CDH11, COL11A1, COL14A1, COL19A1, ICAM1, ITGB1, VCAM 1;
Cell-matrix Adhesion: ITGA1, ITGA2, ITGA3, ITGAM, ITGAV, ITGB1, SPP1;
Other Cell Adhesion Molecules: BGLAP, CD36, COL12A1, COL15A1, COL16A1, COL18A1, COL4A3, COL5A1, COL7A1, COMP, FN1, IBSP, SCARB1, TNF; or
Transcription Factors and Regulators: MSX1, MSX2, NFKB1, RUNX2, SMAD1, SMAD2, SMAD3, SMAD4, SMAD5, SMAD6, SMAD7, SMAD9, SOX9, TNF, TWIST1, TWIST2, VDR.

16. The improvement of claim 15 where locally transferring at least one gene or a gene-based molecule in a plasmid ex vivo comprises locally transferring any molecule and its inhibitor, enhancer, regulator, gene, siRNA, shRNA, antigen, antibody, or peptide which is related to the at least one molecule from the group.

17. An apparatus comprising:
means for applying a low strength electric field network (LSEN) to a donor organ, tissue or cells; and
means for locally transferring at least one gene or a gene-based molecule in a plasmid ex vivo in a time interval between harvest and implantation of an allograft or xenograft before implantation to introduce the long-term over expression of at least one immunosuppressive and/or modulative molecule, or to down regulate at least one alloreactive molecule in the donor organ, tissue or cells only and not in the recipient's whole body system.

18. The apparatus of claim 17 where the means for applying a low strength electric field network (LSEN) to a donor organ, tissue or cells comprises a negative electrode mesh and at least one positive electrode, and a source of low voltage pulsed DC coupled to the network; and
where the means for locally transferring at least one gene or a gene-based molecule in a plasmid ex vivo in a time interval between harvest and implantation of an allograft or xenograft before implantation comprises an infuser or injection device for infusing or injecting the plasmid during application of LSEN to the donor organ, tissue or cells.

19. An apparatus comprising:
a low strength (≦10 v/cm) electric field network (LSEN) arranged and configured to be applied to a whole heart of large animal or human; and
an infuser or injection device of at least one gene into the cells of the whole heart of a large animal or human to induce a plasmid DNA transfer therein during application of the LSEN.
